(19) United States
(12) Patent Application Publication
(10) Pub. No.: US 2019/0345436 A1
(43) Pub. Date: Nov. 14, 2019

(54) PROTEINIC BIOMASS PREPARATION COMPRISING A NON-NATIVE ORGANISM OF THE CLOSTRIDIA CLASS

(71) Applicant: White Dog Labs, Inc., New Castle, DE (US)

(72) Inventors: Bryan P. TRACY, Wilmington, DE (US); Shawn William JONES, Bear, DE (US); Aharon M. EYAL, Jerusalem (IL)

(21) Appl. No.: 16/478,153

(22) PCT Filed: Jan. 16, 2018
(86) PCT No.: PCT/US18/13887

§ 371 Date: Jul. 16, 2019

Related U.S. Application Data
(60) Provisional application No. 62/447,178, filed on Jan. 17, 2017.

(51) Int. Cl. C12N 1/20; A23K 10/18; A23K 20/189; A23K 50/80; C12N 9/12; C12N 9/04; C12N 9/88; C12P 13/08; C12P 13/12; C12P 13/22; C12P 7/26; C12P 7/64; C12P 21/00

(52) U.S. Cl. CPC C12N 1/20; C12N 2500/02; A23K 20/189; A23K 50/80; C12N 9/1217; C12Y 207/02004; C12N 9/0006; C12Y 101/01003; C12N 9/1205; C12Y 207/01039; C12N 9/88; C12Y 401/03027; C12Y 207/0109; C12P 13/08; C12P 13/12; C12P 13/227; C12P 7/26; C12P 7/6427; C12P 21/00; A23K 10/18

(57) ABSTRACT

A proteinic biomass preparation comprising a non-native organism of the Clostridia class, which organism expresses (i) a modified aspartate kinase; (ii) a modified homoserine dehydrogenase; (iii) a modified homoserine kinase; (iv) a modified anthranilate synthase; (v) a functional lycopene pathway and the genes crtY, crtW, and crtZ; and/or (vi) a functional oleic acid pathway and the four gene operon (pfaABCD). Methods of producing proteinic biomass preparations are also described.

### CROSS REFERENCE TO RELATED APPLICATIONS

The instant application claims priority to U.S. Provisional Patent Application No. 62/447,178, filed Jan. 17, 2017, which application is incorporated by reference herein in its entirety.

### FIELD OF THE INVENTION

The field of art to which this invention generally pertains is the production of proteinic biomass preparation comprising a non-native organism of the Clostridia class.

### BACKGROUND

Global demand for high-quality protein for animal and aquaculture feed applications has dramatically grown over the past several decades, leading to increasing costs. There is a particular demand within the aquaculture industry for an alternative protein source, since fishmeal is currently used as the primary protein source. However, fishmeal is not easily replaced because plant-based protein sources (like soy proteins) do not provide the same favorable amino acid profile found in fishmeal. An alternative to both fishmeal and plant-based protein sources is single-cell protein (SCP), where bacterial cell mass provide the protein source.

In order to replace fishmeal with a SCP source, it must mimic key characteristics of fishmeal. Of primary importance is the protein content of the SCP, particularly the amino acid profile with lysine, threonine, methionine, and tryptophan being of high importance. In addition to the protein content and make-up, using a SCP organism capable of producing omega-3 fatty acids is highly desirable. Fish acquire omega-3 fatty acids by consuming microalgae and plankton which are the source of omega-3 fatty acids. If the SCP cannot supply the omega-3 fatty acids, they must be supplemented into the feed from other sources, leading to higher feed costs. Two of the most abundant and important omega-3 fatty acids are eicosapentaenoic acid (EPA) and docosahexaenoic acid (DHA). These are derived from fatty acid biosysnthesis, specifically from oleic acid.

Though not important from a nutrient perspective, astaxanthin, a carotenoid, is important for marketing purposes, as it is responsible for the red color of salmon meat and cooked shellfish. It is part of the terpene family of chemicals and is derived from either the mevalonate pathway or the non-mevalonate (MEP/DOXP) pathway. Addition of an astaxanthin pathway to a SCP host can help cut feed costs as the microorganism itself can produce this feed component, reducing or eliminating the need to add synthetic astaxanthin. Another important addition to aquaculture feed is enzymes to aid digestion of feed components, such as phytases, lipases, and proteases. These enzymes help breakdown the feed components allowing them to be utilized by the fish. For example, phytase removes a phosphate groups from phytic acid allowing the phosphate groups to be uptaken and used by the fish. Phytase hydrolysis also liberates feed minerals complexed by phytic acid, increasing their bioavailability. Engineering a SCP microorganism to natively express these enzymes could further aid in cost reduction of the feed.

### SUMMARY OF THE INVENTION

According to one aspect, provided is a proteinic biomass preparation comprising a non-native organism of the Clostridia class, which organism expresses (i) a modified aspartate kinase characterized by reduced lysine inhibition, reduced threonine inhibition, and/or reduced methionine inhibition compared with the unmodified enzyme in native organism of the same genus and species; (ii) a modified homoserine dehydrogenase characterized by reduced threonine inhibition compared with the unmodified enzyme in native organism of the same genus and species; (iii) a modified homoserine kinase characterized by reduced methionine inhibition compared with the unmodified enzyme in native organism of the same genus and species; (iv) a modified anthranilate synthase characterized by reduced tryptophan inhibition compared with the unmodified enzyme in native organism of the same genus and species; (v) a functional lycopene pathway and the genes crtY, crtW, and crtZ and/or (vi) a functional oleic acid pathway and the four gene operon (pfaABCD).

According to an embodiment, said organism expresses a modified aspartate kinase characterized by reduced lysine inhibition, reduced threonine inhibition, and/or reduced methionine inhibition compared with the unmodified enzyme in native organism of the same genus and species. According to an embodiment, said organism expresses a modified homoserine dehydrogenase characterized by reduced threonine inhibition compared with the unmodified enzyme in native organism of the same genus and species. According to an embodiment, said organism expresses a modified homoserine kinase characterized by reduced methionine inhibition compared with the unmodified enzyme in native organism of the same genus and species. According to an embodiment, said organism expresses a modified anthranilate synthase characterized by reduced tryptophan inhibition compared with the unmodified enzyme in native organism of the same genus and species. According to an embodiment, said organism expresses a functional lycopene pathway and the genes crtY, crtW, and crtZ. According to an embodiment, said organism expresses a functional oleic acid pathway and the four gene operon (pfaABCD). According to an embodiment, said organism further expresses the gene pfaE.

According to an embodiment, at least one of said modified enzymes comprises a spontaneous mutation, a random mutation, site-specific mutation, or a combination thereof. According to an embodiment, at least one of said modified enzymes comprises mutation to the regulatory domain of the enzymes. According to an embodiment, at least one of said modified enzymes comprises mutation to the binding site of lysine, threonine, methionine, and/or tryptophan.

According to an embodiment, amino acid transport occurs at a lower rate in said non-native organism compared with that in a native organism of the same genus and species.

According to an embodiment, said non-native organism is not genetically modified. According to an alternative embodiment, said non-native organism is genetically modified.

According to an embodiment, said non-native organism is selected from Butyribacterium methylotrophicum, Eubacterium limosum, Clostridium kluyveri and combinations thereof. According to an embodiment, said non-native organism is an acetogen. According to an embodiment, said preparation consists of more than one bacterial species. According to an embodiment, said preparation consists of an acetogenic species and a non-acetogenic species.

According to an embodiment, said preparation comprises, on a dry basis, at least 55% wt protein.

According to an embodiment, said preparation comprises, on total protein content, at least 6% wt lysine. According to an embodiment, said preparation comprises, on total protein content, at least 3% wt threonine. According to an embodiment, said preparation comprises, on total protein content, at least 1.5% wt methionine. According to an embodiment, said preparation comprises, on total protein content, at least 0.5% wt tryptophan. According to an embodiment, said preparation comprises, on a dry basis, at least 0.01% wt astaxanthin. According to an embodiment, said preparation comprises, on a dry basis, at least 0.1% wt eicosapentaenoic acid. According to an embodiment, said preparation comprises, on a dry basis, at least 0.1% wt docosahexaenoic acid.

According to an embodiment, said preparation confers a probiotic benefit.

According to an embodiment, said preparation further comprising digestibility-enhancing enzymes selected from the group consisting of phytases, cellulases, lipases, amylases, arabinases, pectinases, mannases, keratinases, proteases, tannases, galactosidases, glucosidases, invertases and combinations thereof. According to an embodiment, said digestibility-enhancing enzymes are generated endogenously by said non-native organism.

According to an embodiment, said non-native organism further expresses a diphosphate-fructose-6-phosphate 1-phosphotransferase (PFP, EC 2.7.1.90). According to an embodiment, phosphofructokinase 1 (EC 2.7.1.11, pfkA, BUME_09340) has been deleted from the genome of said non-native organism. According to an embodiment, acetyl-CoA acetyltransferase gene (thlA, EC 2.3.1.9, BUME_07140) has been deleted from the genome of said non-native organism.

According to an embodiment, further provided is an animal feed comprising said proteinic biomass preparation. According to an embodiment, further provided is a fish feed comprising said proteinic biomass preparation.

Also provided is a method for producing a proteinic preparation comprising culturing said non-native Clostridia class organism in a fermentation medium comprising a carbon source and a nitrogen source, whereby proteinic biomass is generated in a fermentation broth. According to an embodiment, said culturing is anaerobic. According to an embodiment, said fermentation medium comprises stillage. According to an embodiment, said fermentation medium comprises glycerol. According to an embodiment, said fermentation medium comprises CO₂ or a precursor thereof. According to an embodiment, said non-native organism fixes CO₂. According to an embodiment, said fermentation medium further comprises a non-sugar reductant.

According to an embodiment of said method, biomass generation yield is greater than 35 gram (g) biomass per 100 g of carbon source consumed.

Further provided is a proteinic biomass preparation comprising a non-native organism of the Clostridia class modified for expression of peptides and/or proteins, which peptides and/or proteins comprise, on total protein content: (i) at least 6% wt lysine, (ii) at least 3% wt threonine, (iii) at least 1.5%wt methionine, and/or (iv) at least 0.5% wt tryptophan.

Further provided is a proteinic biomass preparation comprising an organism of the Clostridia class, wherein said preparation comprises, (i) on dry basis at least 55% wt protein; (ii) on total protein content, at least 6% wt lysine; (iii) on total protein content, at least 3% wt threonine; (iv) on total protein content, at least 1.5% wt methionine; (v) on total on total protein content, at least 0.5% wt tryptophan; (vi) on a dry basis, at least 0.01% wt astaxanthin; (vii) on a dry basis, at least 0.1% wt eicosapentaenoic acid, and/or (viii) on a dry basis, at least 0.1% wt docosahexaenoic acid. According to an embodiment, said preparation comprises at least two of (i) to (viii). According to an embodiment, said preparation comprises (i) and at one of (ii) to (v). According to an embodiment, said preparation comprises at least one of (vii) and (viii) and at least one of (i) to (v). According to an embodiment, said preparation comprises (vi), at least one of (vii) and (viii) and at least one of (i) to (v).

According to an embodiment, said organism is not genetically modified. According to an alternative embodiment, said organism is genetically modified.

According to an embodiment, said organism expresses (i) a modified aspartate kinase characterized by reduced lysine inhibition, reduced threonine inhibition, and/or reduced methionine inhibition compared with the unmodified enzyme in native organism of the same genus and species; (ii) a modified homoserine dehydrogenase characterized by reduced threonine inhibition compared with the unmodified enzyme in native organism of the same genus and species; (iii) a modified homoserine kinase characterized by reduced methionine inhibition compared with the unmodified enzyme in native organism of the same genus and species; (iv) a modified anthranilate synthase characterized by reduced tryptophan inhibition compared with the unmodified enzyme in native organism of the same genus and species; (v) a functional lycopene pathway and the genes crtY, crtW, and crtZ and/or (vi) a functional oleic acid pathway and the four gene operon (pfaABCD). According to an embodiment, said organism further expresses the gene pfaE.

According to an embodiment, at least one of said modified enzymes comprises a spontaneous mutation, a random mutation, site-specific mutation, or a combination thereof. According to an embodiment, at least one of said modified enzymes comprises mutation to the regulatory domain of the enzymes. According to an embodiment, at least one of said modified enzymes comprises mutation to the binding site of lysine, threonine, methionine and/or tryptophan.

According to an embodiment, said non-native organism further expresses a diphosphate-fructose-6-phosphate 1-phosphotransferase (PFP, EC 2.7.1.90). According to an embodiment, phosphofructokinase 1 (EC 2.7.1.11, pfkA, BUME_09340) has been deleted from the genome of said non-native organism. According to an embodiment,acetyl-CoA acetyltransferase gene (thlA, EC 2.3.1.9, BUME_07140) has been deleted from the genome of said non-native organism.

According to an embodiment, said organism amino acid transport rate is less than the amino acid transport rate in the native form of the organism.

According to an embodiment, said organism is selected from Butyribacterium methylotrophicum, Eubacterium limosum, and Clostridium kluyveri. According to an embodiment, said organism is an acetogen. According to an embodiment, said preparation consists of more than one bacterial species. According to an embodiment, said preparation consists of an acetogenic species and a non-acetogenic species.

According to an embodiment, said preparation confers a probiotic benefit. According to an embodiment, said preparation, further comprises digestibility-enhancing enzymes selected from the group consisting of phytases, cellulases, lipases, amylases, arabinases, pectinases, mannases, keratinases, proteases, tannases, galactosidases, glucosidases, invertases and combinations thereof. According to an embodiment, said digestibility-enhancing enzymes are generated endogenously by said non-native organism.

According to an embodiment, further provided is an animal feed comprising said preparation. According to an embodiment, further provided is fish feed comprising said preparation.

According to an embodiment, further provided is a method for producing of a biomass comprising culturing said organism in a fermentation medium comprising a carbon source and a nitrogen source, whereby biomass is generated in a fermentation broth. According to an embodiment, further provided is said culturing is anaerobic. According to an embodiment, said fermentation medium comprises stillage. According to an embodiment, said fermentation medium comprises glycerol. According to an embodiment, said fermentation medium comprises CO₂ or a precursor thereof. According to an embodiment, said non-native organism fixes CO₂.

According to an embodiment, said fermentation medium further comprises a non-sugar reductant. According to an embodiment, biomass generation yield is greater than 35 g biomass per 100 g of carbon source consumed.

### BRIEF DESCRIPTION OF THE DRAWINGS

FIG. 1 depicts an exemplary co-location integrated method for producing ethanol.

FIG. 2 shows exemplary results of B. methylotrophicum fermentation on glucose.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein the term proteinic biomass refers to biomass comprising at least 50% protein.

As used herein the term comprising an amino acid refers to either comprising the amino acid in its free form or comprising peptides or proteins, the hydrolysate of which comprises that amino acid.

As used herein, the term genetically modified organisms refers to organism comprising specific modifications to the genome. These can include chromosomal deletions or insertions and expression of exogenous genes on a replicating plasmid. As used herein, the term genetic modifications does not refer to single point mutations or mutations arising from adaptation experiments or induced mutatgenesis experiments. As used herein, the term non-genetically modified organisms includes organisms comprising single point mutations or mutations arising from adaptation experiments or induced mutatgenesis experiments.

According to one aspect, provided is a proteinic biomass preparation comprising a non-native organism of the Clostridia class, which organism expresses (i) a modified aspartate kinase characterized by reduced lysine inhibition, reduced threonine inhibition, and/or reduced methionine inhibition compared with the unmodified enzyme in native organism of the same genus and species; (ii) a modified homoserine dehydrogenase characterized by reduced threonine inhibition compared with the unmodified enzyme in native organism of the same genus and species; (iii) a modified homoserine kinase characterized by reduced methionine inhibition compared with the unmodified enzyme in native organism of the same genus and species; (iv) a modified anthranilate synthase characterized by reduced tryptophan inhibition compared with the unmodified enzyme in native organism of the same genus and species; (v) a functional lycopene pathway and the genes crtY, crtW, and crtZ and/or (vi) a functional oleic acid pathway and the four gene operon (pfaABCD).

According to an embodiment, said organism expresses a modified aspartate kinase characterized by reduced lysine inhibition, reduced threonine inhibition, and/or reduced methionine inhibition compared with the unmodified enzyme in native organism of the same genus and species.

According to an embodiment, native organisms of the Clostridia class can express multiple aspartase kinases, some of which can be inhibited by lysine alone, some by threonine alone, some by methionine and some by their combination. According to various embodiments, said preparation non-native organism expresses a modified aspartate kinase characterized by reduced lysine inhibition compared with native aspartate kinase in native organism; a modified aspartate kinase characterized by reduced threonine inhibition compared with native aspartate kinase in native organism; a modified aspartate kinase characterized by reduced methionine inhibition compared with native aspartate kinase in native organism or a modified aspartate kinase characterized by reduced inhibition by multiple of said amino acids compared with native aspartate kinase in native organism.

According to an embodiment, said modified aspartate kinase is derived from the Butyribacterium methylotrophicum aspartate kinase and is characterized by reduced lysine inhibition, reduced threonine inhibition, and/or reduced methionine inhibition compared with the unmodified Butyribacterium methylotrophicum aspartate kinase. According to an embodiment, the gene of said aspartate kinase is selected from the group consisting of lysC1 (BUME_01940), lysC2 (BUME_01950), and lysC3 (BUME_08600).

According to an embodiment, said organism expresses a modified homoserine dehydrogenase characterized by reduced threonine inhibition compared with the unmodified enzyme in native organism of the same genus and species.

According to an embodiment, said modified homoserine dehydrogenase is derived from the Butyribacterium methylotrophicum homoserine dehydrogenase and is characterized by reduced threonine inhibition compared with the unmodified Butyribacterium methylotrophicum homoserine dehydrogenase. According to an embodiment, the gene of said homoserine dehydrogenase is hom (BUME_08590).

According to an embodiment, said organism expresses a modified homoserine kinase characterized by reduced methionine inhibition compared with the unmodified enzyme in native organism of the same genus and species.

According to an embodiment, said modified homoserine kinase is derived from the Butyribacterium methylotrophicum homoserine kinase and is characterized by reduced methionine inhibition compared with the unmodified Butyribacterium methylotrophicum homoserine kinase. According to an embodiment, the gene of said homoserine kinase is selected from the group consisting of thrB1 (BUME_06990) and thrB2 (BUME_08570).

According to an embodiment, said organism expresses a modified anthranilate synthase characterized by reduced tryptophan inhibition compared with the unmodified enzyme in native organism of the same genus and species.

According to an embodiment, said modified anthranilate synthase is derived from the Butyribacterium methylotrophicum anthranilate synthase and is characterized by reduced tryptophan inhibition compared with the unmodified Butyribacterium methylotrophicum anthranilate synthase. According to an embodiment, the gene of said anthranilate synthase is trpEG (BUME_17910-BUME_17900).

According to an embodiment, at least one of said modified enzymes comprises a spontaneous mutation, a random mutation, site-specific mutation, or a combination thereof. According to an embodiment, at least one of said modified enzymes comprises mutation to the regulatory domain of the enzymes. According to an embodiment, at least one of said modified enzymes comprises mutation to the binding site of lysine, threonine, methionine, and/or tryptophan.

According to an embodiment, said non-native organism expresses a functional lycopene pathway. According to an embodiment, said non-native organism is modified to express a functional lycopene pathway. According to an embodiment, said non-native organism expresses a functional lycopene pathway and the genes crtY, crtW, and crtZ.

According to an embodiment, said organism expresses a functional oleic acid pathway and the four gene operon (pfaABCD). According to an embodiment, said organism further expresses the gene pfaE.

According to an embodiment, said non-native organism further expresses a diphosphate-fructose-6-phosphate 1-phosphotransferase (PFP, EC 2.7.1.90). According to an embodiment, phosphofructokinase 1 (EC 2.7.1.11, pfkA, BUME_09340) has been deleted from the genome of said non-native organism.

According to an embodiment, acetyl-CoA acetyltransferase gene (th1A, EC 2.3.1.9, BUME_07140) been deleted from the genome of said non-native organism.

According to an embodiment, said preparation confers a probiotic benefit. According to an embodiment, said preparation can help disrupt the propagation of pathogenic gut bacteria, thus conferring a probiotic benefit. According to an embodiment, said preparation can induce a positive host response within the gut, thus conferring a probiotic benefit.

According to an embodiment, said preparation comprises enzymes capable of assisting the digestibility of feed ingredients. According to an embodiment, said preparation comprises digestibility-enhancing enzymes selected from the group consisting of phytases, cellulases, lipases, amylases, arabinases, pectinases, mannases, keratinases, proteases, tannases, galactosidases, glucosidases, invertases and combinations thereof. According to an embodiment, said digestibility-enhancing enzymes are generated at least partially endogenously by said non-native organism.

According to an embodiment, amino acid transport occurs at a lower rate in the non-native organism than in a native organism of the same genus and species, e.g. at less than 50% the rate in the native organism, less than 30%, less than 20%, less than 10% or less than 5%. According to an embodiment, said lower rate transport is out of the cell, into the cell or both. According to an embodiment, said lower rate is for transport of intracellular amino acids into the extracellular environment. According to an embodiment, said lower rate is a result of a modification to a Basic Amino Acid Antiporter (ArcD)-family protein. According to an embodiment, lysine transport occurs at a lower rate in the non-native organism. According to an embodiment, threonine transport occurs at a lower rate in the non-native organism. According to an embodiment, tryptophan transport occurs at a lower rate in the non-native organism. According to an embodiment, methionine transport occurs at a lower rate in the non-native organism. According to an embodiment, transport of multiple amino acids occurs at a lower rate in the non-native organism.

According to an embodiment, said non-native organism is not genetically modified.

According to an alternative embodiment, said non-native organism is genetically modified.

According to an embodiment, said non-native organism is selected from Butyribacterium methylotrophicum, Eubacterium limosum, Clostridium kluyveri, Selenomonas bovis, Selenomonas ruminantium subsp. Lactilytica, Selenomonas ruminantium subsp. Ruminantium, Prevotella albensis, Prevotella bryantii, Prevotella brevi, and Megasphaera elsdenii. According to an embodiment, said non-native organism is an acetogen.

According to an embodiment, said preparation consists of more than one bacterial species. According to an embodiment, said preparation consists of an acetogenic species and a non-acetogenic species.

According to an embodiment, said preparation comprises, on a dry basis, at least 55% wt protein, at least 58% wt, at least 60% wt, at least 62% wt, at least 64% wt, at least 66% wt, at least 68% wt, at least 70% wt, at least 72% wt, or at least 74% wt.

According to an embodiment, said preparation comprises, on total protein content, at least 6% wt lysine, at least 7% wt, at least 8% wt, at least 9% wt, at least 10% wt, at least 11% wt, at least 12% wt, at least 13% wt, at least 14% wt, or at least 15% wt.

According to an embodiment, said preparation comprises, on total protein content, at least 3% wt threonine, at least 3.5% wt, at least 4% wt, at least 4.5% wt, at least 5% wt, at least 5.5% wt or at least 6% wt.

According to an embodiment, said preparation comprises, on total protein content, at least 1.5% wt methionine, at least 1.7% wt, at least 1.8% wt, at least 1.9% wt, at least 2% wt, at least 2.1% wt, at least 2.2% wt, at least 2.3% wt, at least 2.4% wt or at least 2.5% wt.

According to an embodiment, said preparation comprises, on total protein content, at least o.5% wt tryptophan, at least 0.7% wt, at least 0.8% wt, at least 0.9% wt, at least 1% wt, at least 1.1% wt, at least 1.2% wt, at least 1.3% wt, at least 1.4% wt or at least 1.5% wt.

According to an embodiment, said preparation comprises, on a dry basis, at least 0.01% wt astaxanthin, at least 0.02% wt, at least 0.03% wt, at least 0.04% wt, at least 0.05% wt, at least 0.06% wt, at least 0.07% wt, at least 0.08% wt, at least 0.09% wt, least 0.1% wt, at least 0.11% wt, at least 0.12% wt, least 0.13% wt, at least 0.14% wt or least 0.15% wt.

According to an embodiment, said preparation comprises, on a dry basis, at least 0.1% wt eicosapentaenoic acid, at least 0.2% wt, at least 0.3% wt, at least 0.4% wt, at least 0.5% wt, at least 0.6% wt, at least 0.7% wt, at least 0.8% wt, at least 0.9% wt, least 1.0% wt, at least 1.1% wt, at least 1.2% wt, least 1.3% wt, at least 1.4% wt or least 1.5% wt.

According to an embodiment, said preparation comprises, on a dry basis, at least 0.1% wt docosahexaenoic acid, at least 0.2% wt, at least 0.3% wt, at least 0.4% wt, at least 0.5% wt, at least 0.6% wt, at least 0.7% wt, at least 0.8% wt, at least 0.9% wt, least 1.0% wt, at least 1.1% wt, at least 1.2% wt, least 1.3% wt, at least 1.4% wt or least 1.5% wt.

According to an embodiment, further provided is an animal feed comprising said proteinic biomass preparation. According to an embodiment, further provided is fish feed comprising said proteinic biomass preparation.

Also provided is a method for producing a proteinic preparation, which method comprises culturing said non-native Clostridia class organism in a fermentation medium comprising a carbon source and a nitrogen source, whereby said proteinic biomass is generated in a fermentation broth. According to an embodiment, said method further comprises separating said generated biomass from the fermentation medium. According to an embodiment, said separating comprises at least one of filtering and centrifugation and optionally washing said separated cells in order to wash off water-soluble compounds, such as ashes and carboxylic acid salts. According to an embodiment, said method further comprises at least one of lysing said biomass and drying it.

According to an embodiment, said fermentation broth further comprises a coproduct selected from the group consisting of acetic acid, butyric acid, lactic acid, ethanol, n-butanol, 1,3-propanediol, 2,3-butanediol, acetoin and combinations thereof. According to an embodiment, said method further comprises separating said coproduct from said fermentation broth. According to an embodiment, said separating comprises adjusting the pH of said broth to pH<4.

According to an embodiment, said culturing is anaerobic.

According to an embodiment, said fermentation medium comprises stillage. According to various embodiments, said stillage in whole stillage, thin stillage, combinations thereof or products thereof. According to an embodiment, said fermentation medium comprises glycerol.

According to an embodiment, said fermentation medium further comprises CO₂ or a precursor thereof. According to an embodiment, said method comprises sparging CO₂ through said medium and/or adding there a carbonate or a bicarbonate (e.g. sodium carbonate or sodium bicarbonate). According to an embodiment, said cultured organism fixes CO₂.

According to an embodiment, said fermentation medium further comprises a non-sugar reductant.

According to an embodiment, in said method biomass generation yield is greater than 35 g biomass per 100 g of carbon source consumed greater than 40 g, greater than 45 g, greater than 50 g, or greater than 55 g. According to an embodiment, cell density in said fermentation broth is at least 15 gram cell mass per Liter (15 g/L), at least 20 g/L, at least 25 g/L, at least 30 g/L, at least 35 g/L or at least 40 g/L. According to an embodiment, cell culturing productivity in said fermentation broth is at least 0.5 gram/Liter/hour (g/L/hr), at least 0.6 g/L/hr, at least 0.7 g/L/hr, at least 0.8 g/L/hr, at least 0.9 g/L/hr, at least 1.0 g/L/hr, at least 1.1 g/L/hr, at least 1.2 g/L/hr or at least 1.3 g/L/hr.

According to an embodiment, said method further comprises combining said biomass, optionally lysed and/or dried, with other feed ingredients, such as fishmeal, fishoil, other animal proteins, other vegetable proteins, vitamins and/or minerals. According to an embodiment, said method further comprises pelletizing.

According to another aspect, provided is a proteinic biomass preparation comprising a non-native organism of the Clostridia class modified for expression of peptides and/or proteins, which peptides and/or proteins comprise, on a total protein content: (i), at least 6% wt lysine, at least 7% wt, at least 8% wt, at least 9% wt, at least 10% wt, at least 11% wt, at least 12% wt, at least 13% wt, at least 14% wt, or at least 15% wt; (ii) at least 3% wt threonine, at least 3.5% wt, at least 4% wt, at least 4.5% wt, at least 5% wt, at least 5.5% wt or at least 6% wt; (iii) at least 1.5% wt methionine, at least 1.7% wt, at least 1.8% wt, at least 1.9% wt, at least 2% wt, at least 2.1% wt, at least 2.2% wt, at least 2.3% wt, at least 2.4% wt or at least 2.5% wt; and/or (iv) at least 0.5% wt tryptophan at least 0.7% wt, at least 0.8% wt, at least 0.9% wt, at least 1% wt, at least 1.1% wt, at least 1.2% wt, at least 1.3% wt, at least 1.4% wt or at least 1.5% wt.

According to an embodiment, protein content and amino acid profile is modified by expression of a peptide sequence. This sequence can be a native peptide, an exogenous peptide, or a synthetic peptide sequence. The resulting peptide can be water insoluble.

According to another aspect, provided is proteinic biomass comprising an organism of the Clostridia class, wherein said preparation comprises, (i) on dry basis at least 55% wt protein, at least 58% wt, at least 60% wt, at least 62% wt, at least 64% wt, at least 66% wt, at least 68% wt, at least 70% wt, at least 72% wt, or at least 74% wt; (ii) on total protein content, at least 6% wt lysine at least 7% wt, at least 8% wt, at least 9% wt, at least 10% wt, at least 11% wt, at least 12% wt, at least 13% wt, at least 14% wt, or at least 15% wt; (iii) on total protein content, at least 3% wt threonine, at least 3.5% wt, at least 4% wt, at least 4.5% wt, at least 5% wt, at least 5.5% wt or at least 6% wt; (iv) on total protein content, at least 1.5% wt methionine, at least 1.7% wt, at least 1.8% wt, at least 1.9% wt, at least 2% wt, at least 2.1% wt, at least 2.2% wt, at least 2.3% wt, at least 2.4% wt or at least 2.5% wt; (v) on total on total protein content, at least 0.5% wt tryptophan, at least 0.7% wt, at least 0.8% wt, at least 0.9% wt, at least 1% wt, at least 1.1% wt, at least 1.2% wt, at least 1.3% wt, at least 1.4% wt or at least 1.5% wt; (vi) on a dry basis, at least 0.01% wt astaxanthin, at least 0.02% wt, at least 0.03% wt, at least 0.04% wt, at least 0.05% wt, at least 0.06% wt, at least 0.07% wt, at least 0.08% wt, at least 0.09% wt, least 0.1% wt, at least 0.11% wt, at least 0.12% wt, least 0.13% wt, at least 0.14% wt or least 0.15% wt; (vii) on a dry basis, at least 0.1wt eicosapentaenoic acid, at least 0.2% wt, at least 0.3% wt, at least 0.4% wt, at least 0.5% wt, at least 0.6% wt, at least 0.7% wt, at least 0.8% wt, at least 0.9% wt, least 1.0% wt, at least 1.1% wt, at least 1.2% wt, least 1.3% wt, at least 1.4% wt or least 1.5% wt; and/or (viii) on a dry basis, at least 0.1% wt docosahexaenoic acid, at least 0.2% wt, at least 0.3% wt, at least 0.4% wt, at least 0.5% wt, at least 0.6% wt, at least 0.7% wt, at least 0.8% wt, at least 0.9% wt, least 1.0% wt, at least 1.1% wt, at least 1.2% wt, least 1.3% wt, at least 1.4% wt or at least 1.5% wt.

According to various embodiment, said proteinic biomass comprises at least two of (i) to (viii), at least three, at least four, at least five, at least six, at least seven or all eight.

According to various embodiment, said proteinic biomass comprises (i) and at one of (ii) to (v), at least two, at least three or all four.

According to various embodiment, said proteinic biomass comprises (vi) and at one of (i) to (v), at least two, at least three, at least four or all five.

According to various embodiment, said proteinic biomass comprises at least one of (vii) and (viii) and at one of (i) to (v), at least two, at least three, at least four or all five.

According to various embodiment, said proteinic biomass comprises (vi); at least one of (vii) and (viii) and at one of (i) to (v), at least two, at least three, at least four or all five.

According to an embodiment, said organism is not genetically modified. According to an alternative embodiment, said organism is genetically modified.

According to an embodiment, said organism expresses (i) a modified aspartate kinase characterized by reduced lysine inhibition, reduced threonine inhibition, and/or reduced methionine inhibition compared with the unmodified enzyme in native organism of the same genus and species; (ii) a modified homoserine dehydrogenase characterized by reduced threonine inhibition compared with the unmodified enzyme in native organism of the same genus and species; (iii) a modified homoserine kinase characterized by reduced methionine inhibition compared with the unmodified enzyme in native organism of the same genus and species; (iv) a modified anthranilate synthase characterized by reduced tryptophan inhibition compared with the unmodified enzyme in native organism of the same genus and species; (v) a functional lycopene pathway and the genes crtY, crtW, and crtZ and/or (vi) a functional oleic acid pathway and the four gene operon (pfaABCD).

According to an embodiment, said modified aspartate kinase is derived from the Butyribacterium methylotrophicum aspartate kinase and is characterized by reduced lysine inhibition, reduced threonine inhibition, and/or reduced methionine inhibition compared with the unmodified Butyribacterium methylotrophicum aspartate kinase. According to an embodiment, the gene of said aspartate kinase is selected from the group consisting of lysC1 (BUME_01940), lysC2 (BUME_01950), and lysC3 (BUME_08600).

According to an embodiment, said modified homoserine dehydrogenase is derived from the Butyribacterium methylotrophicum homoserine dehydrogenase and is characterized by reduced threonine inhibition compared with the unmodified Butyribacterium methylotrophicum homoserine dehydrogenase. According to an embodiment, the gene of said homoserine dehydrogenase is hom (BUME_08590).

According to an embodiment, said modified homoserine kinase is derived from the Butyribacterium methylotrophicum homoserine kinase and is characterized by reduced methionine inhibition compared with the unmodified Butyribacterium methylotrophicum homoserine kinase. According to an embodiment, the gene of said homoserine kinase is selected from the group consisting of thrB1 (BUME_06990) and thrB2 (BUME_08570).

According to an embodiment, said modified anthranilate synthase is derived from the Butyribacterium methylotrophicum anthranilate synthase and is characterized by reduced tryptophan inhibition compared with the unmodified Butyribacterium methylotrophicum anthranilate synthase. According to an embodiment, the gene of said anthranilate synthase is trpEG (BUME_17910-BUME_17900).

According to an embodiment, at least one of said modified enzymes comprises a spontaneous mutation, a random mutation, site-specific mutation, or a combination thereof. According to an embodiment, at least one of said modified enzymes comprises mutation to the regulatory domain of the enzymes. According to an embodiment, at least one of said modified enzymes comprises mutation to the binding site of lysine, threonine, methionine, and/or tryptophan.

According to an embodiment, amino acid transport occurs at a lower rate in the non-native organism than in a native organism of the same genus and species, e.g. at less than 50% the rate in the native organism, less than 30%, less than 20%, less than 10% or less than 5%. According to an embodiment, said lower rate transport is out of the cell, into the cell or both. According to an embodiment, said lower rate is for transport of intracellular amino acids into the extracellular environment. According to an embodiment, said lower rate is a result of a modification to a Basic Amino Acid Antiporter (ArcD)-family protein.

According to an embodiment, said non-native organism is selected from Butyribacterium methylotrophicum, Eubacterium limosum, Clostridium kluyveri, Selenomonas bovis, Selenomonas ruminantium subsp. Lactilytica, Selenomonas ruminantium subsp. Ruminantium, Prevotella albensis, Prevotella bryantii, Prevotella brevi, and Megasphaera elsdenii. According to an embodiment, said non-native organism is an acetogen.

According to an embodiment, further provided is an animal feed comprising said proteinic biomass preparation. According to an embodiment, further provided is fish feed comprising said proteinic biomass preparation.

According to an embodiment, further provided is a method for producing a proteinic biomass preparation comprising culturing said non-native Clostridia class organism in a fermentation medium comprising a carbon source and a nitrogen source, whereby said proteinic biomass is generated in fermentation broth. According to an embodiment, said culturing is anaerobic.

According to an embodiment, said fermentation medium comprises stillage. According to an embodiment, said fermentation medium further comprises a non-sugar reductant.

According to an embodiment, in said method biomass generation yield is greater than 35 g biomass per 100 g of carbon source consumed greater than 40 g, greater than 45 g, greater than 50 g, or greater than 55 g. According to an embodiment, cell density in said fermentation broth is at least 15 g cell mass per Liter (15 g/L), at least 20 g/L, at least 25 g/L, at least 30 g/L, at least 35 g/L or at least 40 g/L. According to an embodiment, cell culturing productivity in said fermentation broth is at least 0.5 gram/Liter/hour (g/L/hr), at least 0.6 g/L/hr, at least 0.7 g/L/hr, at least 0.8 g/L/hr, at least 0.9 g/L/hr, at least 1.0 g/L/hr, at least 1.1 g/L/hr, at least 1.2 g/L/hr or at least 1.3 g/L/hr.

Some embodiments herein, provide methods for producing the proteinic biomass preparation, comprising culturing said non-native Clostridia class organism in a fermentation medium comprising a carbon source and a nitrogen source, whereby said proteinic biomass is generated in a fermentation broth. According to an embodiment, said provided fermentation medium comprises stillage of ethanol production. According to an embodiment, ethanol production includes fermentation of carbohydrates-containing feedstock to form a fermentation broth comprising ethanol, biomass and non-fermented components of the feedstock, e.g. carbon sources and proteins. According to an embodiment, ethanol is distilled out of said broth to form distilled ethanol and a residue comprising said biomass and non-fermented components of the feedstock. This residue is referred to as whole stillage. According to an embodiment, the provided fermentation medium comprises said whole stillage. Alternatively, the whole stillage is filtered or centrifuged to generate wet solids and a solids-depleted liquid referred to as thin stillage. According to an embodiment, the provided fermentation medium comprises said thin stillage. A typical thin stillage contains glycerol at about 36 g/L, glucose, DP2, DP3 and DP4+ at 0.7 g/L, 17 g/L, 5 g/L and 28 g/L, respectively and lactic acid at 2.5 g/L.

Several of the above embodiment, provided methods for producing the proteinic biomass preparation, comprising culturing said non-native Clostridia class organism in a fermentation medium comprising a carbon source and a nitrogen source, whereby said proteinic biomass is generated in a fermentation broth. According to an embodiment said method is conducted at co-location with ethanol production. As used herein, the term co-location refers to location within 10 Km from each other, within 5 Km, within 2 Km or within 1 Km.

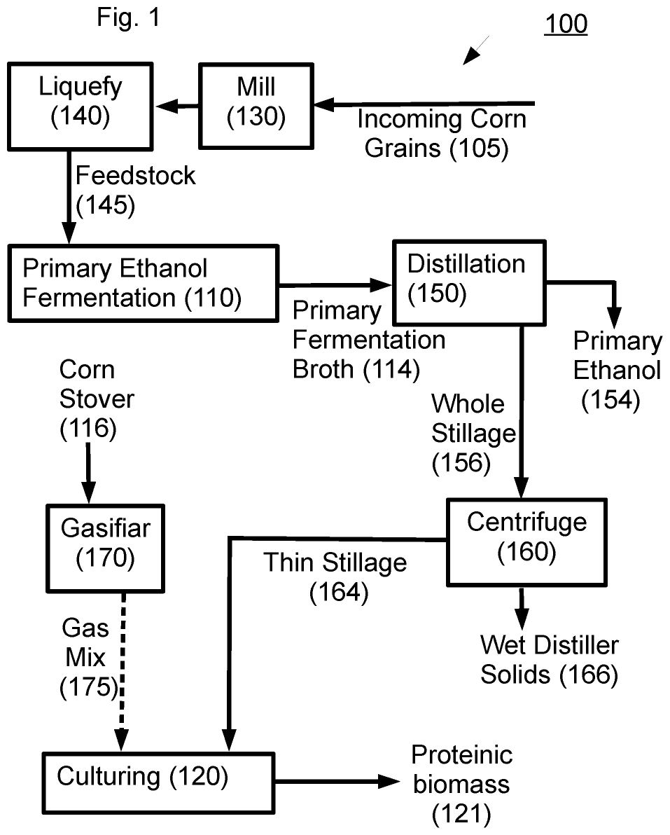

An exemplary co-location integrated method for producing ethanol is depicted in FIG. 1. It comprises, a primary ethanol fermentation [110] generating a primary ethanol stream [154] and stillage [156] and a secondary mixotrophic ethanol fermentation [120], wherein said stillage forms a fraction of the fermentation medium and wherein a secondary ethanol stream [194] is generated. According to an embodiment, the method further comprises milling [130] and liquefying [140] incoming corn grains [105] to form the feedstock [145] of the primary fermentation. According to an embodiment, the method further comprises fractionating the corn grains, e.g. for pre-removal of fiber and/or corn oil (not shown in the figure). The liquefied-corn-containing primary fermentation medium is metabolized by an ethanol producing organism, e.g. a yeast, in [110]. A primary fermentation broth is formed [114] containing ethanol. In distillation columns [150], ethanol is distilled out, forming a primary ethanol stream [154], which is optionally further dried on molecular sieves (not shown in the figure). The residue [156] is the whole stillage comprising the yeast, corn protein, optionally also fiber and oil, and soluble matter including glycerol and oligosaccharides. The whole stillage is centrifuged [160] to form wet distillers solids [166] and thin stillage [164].

The exemplary method further comprises gasification of corn stover [116] in a gasifier [170] to form a mixture of hydrogen, CO and CO2 [175] to be used as non-sugar reductant. Said non-sugar reductant is combined with said thin stillage (the carbon source) to form the feedstock for the fermentation [120] medium, wherein said non-native Clostridia class organism is cultured and whereby proteinic biomass is generated in a fermentation broth [121]. Optionally, said biomass is separated, dried and lysed (not shown in the figure).

### EXAMPLES

### Example 1—Fermentation of Butyribacterium Methylotrophicum

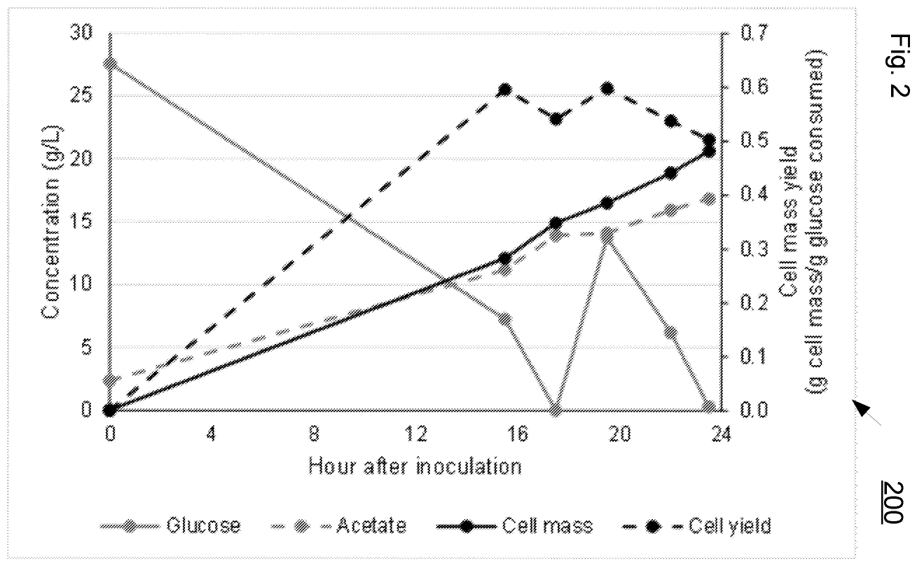

A 3-L batch fermentation was conducted with Butyribacterium methylotrophicum grown on glucose. The fermenter was inoculated with a 10% (v/v) inoculum of an actively growing culture. The medium in the fermenter consisted of 0.2 g/L of K₂HPO₄.3H₂O, 0.3 g/L of KH₂PO₄, 0.3 g/L of (NH₄)₂SO₄, 0.6 g/L of NaCl, 0.12 g/L of MgSO₄.7H₂O, 0.1 g/L of CaCl₂.2H₂O, 0.5 g/L of cysteine HCl, 1 g/L yeast extract, 3 g/L sodium acetate, 30 g/L of glucose, 10 mL/L Wolfe's Mineral Solution, and 10 mL/L Wolfe's Vitamin Solution. The fermenter was sparged with N₂ until just after inoculation, at which time the sparging was turned off. The pH was bottom controlled at 6.5 with 6M NH₄OH. Temperature was maintained at 37° C. with agitation of 100 rpm. At 19.5 hours after inoculation, the culture was fed another ˜14 g/L of glucose, as the culture was exhausted of glucose (FIG. 2).

The cell density reached over 20 g/L in the first 24 hours of fermentation with over 40 g/L of glucose consumed and about 14.5 g/L of acetate being produced. The cell mass yield was consistently over 0.5 g/g after 12 hours of growth.

### Example 2—Reduced Inhibition of Aspartate Kinase

Butyribacterium methylotrophicum has three annotated aspartate kinase genes: lysC1 (BUME_01940), lysC2 (BUME_01950), and lysC3 (BUME_08600).

BUME_01940 (amino acid sequence)
MTTTYMESHSVDGLTVDHKNLMISLKKVPVNSIILTRCLSELSDADVNV
DIITQTAPVKNAFDVSFIVLERDLEKVKDIVNALGEEYPEIKITINKDI
TRLSVSGIGMRTQSGVAAKFFQVLADNDVQILMITTSEIRISCIIKIED
TEKAVAATKEAFDLED
BUME_01950 (amino acid sequence)
MSIIVQKYGGTSMGTIDRIKNVARRIIKKREDGNQMVVVVSAMGKSTDE
LVKMAYSISDAPPRRELDMLLATGEQVSISMLSMALNAMGYDAISFTGP
QVGVHTMGHHGKSRIMDIETRKIEDALNDGKIVIIAGFQGVNENDDITT
LGRGGSDTSAVALSCVLECPCEIYTDVDGIYGVDPRLYPPAKKLDTVSF
DEMLEMASLGAGVMHARAIELGSKYNAEIYVASSIHDVPGTLIKEGDSN
MSPMEQQAITGLAIDNDELMVSLKNVPFDMNITAQFFSDLAKKSINIDM
ISQTAPVYGAINISFSAPIEDLSELRKILYDFMEKYPQVEMDINKEISK
LSVVGIGMRSQSGVAAKFFQLLADNNIPMLMITTSEIRISCVIPSELRD
TAVMATADAFDL
BUME_08600 (amino acid sequence)
MEDLIVQKYGGTSVGTVEKIKRVARRIVETKNAGNKVVVVVSAMGKTTD
ELVDLALAINPNPPSREMDVLLATGEQVSISLLAMAIQTIGHDVVSLTG
AQCGIQTSDVHKRARISGIDTARIERELADEKIVIVAGFQGVDENRDIT
TLGRGGSDTSAVAIAAALEAKCCEIYTDVDGVYNADPRVVPTATKMDEV
SYQEVLEMASLGAGVLHPRSVELAEKFKMPLIVRSSYNNNEGTIIKEDV
KMEKVLVRGIALDENIAKISIFEVPDQPGIAFKLFSMLASANIHVDMIV
QNVNRTAVNDISFTVDADELQEAVEVSQKFAFEVEAQKVAFDKGVAKLS
VVGTGIVANAEIASKFFESLFELGINIQTISTSEIKISCLIDKERAKEA
MIHIHKKFDM

One or more of these genes are subjected to mutagenesis, such as chemically-induced random mutagenesis or error-prone PCR amplification, and then screened for reduced inhibition by lyseine, threonine, and/or methionine.

### Example 3—Reduced Inhibition of Homoserine Dehydrogenase

Butyribacterium methylotrophicum has one annotated homoserine dehydrogenase gene: hom (BUME_08590).

BUME_08590 (amino acid sequence)
MNIGLLGFGTIGTGVYELINLNKGRFAKNLDEKVVITKILDKDPNKKVA
EEDKVARVVTNPDDIMDDPEIEIVIALLGGMDFEYGLIKRALQSGKHVV
TANKAVISEYFEELLTIAAENNVILRYEASVGGGIPIIGSLKEELKINR
VNEIKGILNGTTNFILSKMTEEGADFADTLKLAQSIGFAEADPTADIEG
YDVSRKLAILSSLAYGGIIKDEDVRKRGLSDVRAVDIEMAGDYGYIIKY
LGHSVLKEGNQVYTTVEPVMFKEASIMSNVNSEFNIISIVGDIIGELQF
YGKGAGKDATANAVVGDALYIINCIKDNNFPKPLVFRKQLDKKGVGAFK
GKYYLRVDIDSHETFEHALNAVDEVCARKNIIVSDNRVFFMTEPVEADV
FDAMVAKIKEKQSECFYARIYE

This gene is subjected to mutagenesis, such as chemically-induced random mutagenesis or error-prone PCR amplification, and then screened for reduced inhibition by threonine.

### Example 4—Reduced Inhibition of Homoserine Kinase

Butyribacterium methylotrophicum has two annotated homoserine kinase genes: thrB1 (BUME_06990) or thrB2 (BUME_08570).

BUME_06990 (amino acid sequence)
MVDGKFIEMQTEIMKNYPPANAQALLNIFNAASGMQEYLDDVLAKTRES
YLYTQLRDLVENYYDIGTLLDVYQIFGGYINTSFGIYTEKNGEKQTWFV
RKYKNGKELESLLFEHSMLKYARENGFSYGAVPIPAKDGKTYHEVTETT
TEGETKSYFAVFNFVGGKAHYDWIPNWANAGVADLTVTSAAKSLAEFHN
STRGFDPEGRHGDNIMDNEDITVNEIIRKFPKTLKEYRKSYAEAGFENV
YTEYYDANYDYFAKMCERSVIPDADYNTMVSNVCHCDFHPGNFKYLDNG
EVCGSFDYDMAKIDSRLFELGLAIHYCFSSWLSDTNGIINLGRASLFVK
TYDEELHKAGGIEPLTAIEKKYLYEVTIQGALYDLGWCSSACVYDSTLD
PYEYLFYTQHFVACLKWLEANEEAFRKAFK
BUME_08570 (amino acid sequence)
MIKVRVPATTANIGPGFDAFGMAFQLYNIFSFEERDNGKLTIRGVERRY
QGKSNLVYKAMLKVFNRVHYRPKGIYIYTDVNIPVSRGLGSSAACIVGG
LVGANTLCGAPLTGKELFDMAVEMEGHPDNVAPAMFGGLVVSLGLKEEN
HYIKKEVSQCFEFYGLIPDFTLSTMEARKALPKKVFHKDAVFNVSRATM
TYLALTEGRPDILKVSVEDKLHQPYREGLIAHYDEVSQKARELGALNTC
ISGAGPTLLAITTRDNDQFYAEMGKYLKEKLPGWTLLKLEPDNTGVCTD
QHS

One or more of these genes are subjected to mutagenesis, such as chemically-induced random mutagenesis or error-prone PCR amplification, and then screened for reduced inhibition by methionine.

### Example 5—Reduced Inhibition of Anthranilate Synthase

Butyribacterium methylotrophicum has one annotated anthranilate synthase consisting of two components: trpEG (BUME_17910-BUME_17900).

BUME_17910 (amino acid sequence)
MKIPSLETVQRQSEGFAAFPVSCEIFADIKTPIQVLKILKSISSRCYLLE
SVEGVEKWGRYSFLGFDPVVEVKCKDGLMEIKNGTAVRLETDDPGQEIRR
ILSEYRSPKVAELPPFTGGFVGYFSYDYLKYSEPSLRFDGDDSAGFNDLD
LMLFEKVIAFDQLRQKIVIMVTIKTDHLAVNYNRAVRELDYLVGLIHSDV
PASGQLPRLLSDFKPHFDREAYCEIVKKTKGYIREGDIFQAVPSNRLTAE
MEGSLFNTYRVLRTINPSPYMYYIACDDLEIAGASPETLVKLQDGELSTF
PIAGTSPRGRTSEEDAELEERLLKDPKELAEHNMLVDLGRNDLGRVSRYG
SVKVQEYLKVQKYSHVMHITSVVTGQLRENMDQLDAVTAVLPAGTLSGAP
KIRACEIINELEGIRRGIYGGAIGYIDFTGNMDVCIAIRTAVKKDGRVYV
QSGGGVVADSDPEKEYQESINKAMAVVEAVKQSVEVMD
BUME_17900 (amino acid sequence)
MILIIDNYDSFSYNLVQQVGRVNPDLKVIRNDALSVDEIEALHPSHIILS
PGPGRPADAGVCEAVVRRFSGKLPVLGVCLGHQAICEAFGAEVTYASELI
HGKRSSIHIANGSPLFKGLPPIMDAARYHSLAVSRSSLPDELLIIAEDNA
DEVMGVKHRDFDVYGLQFHPESILTPQGNIIIENFLALGGKIQ

One or more of these genes are subjected to mutagenesis, such as chemically-induced random mutagenesis or error-prone PCR amplification, and then screened for reduced inhibition by tryptophan

### Example 6—Expression of Exogenous Peptide Sequences

In order to modify the protein content or amino acid profile of Butyribacterium methylotrophicum exogenous peptide sequences are expressed to change the composition of the prepared biomass. Examples of exogenous peptide sequences are:

### Glb1

Glb1
MVSARIVVLLAVLLCAAAAVASSWEDDNHHHHGGHKSGRCVRRCEDRPWH
QRPRCLEQCREEEREKRQERSRHEADDRSGEGSSEDEREREQEKEEKQKD
RRPYVFDRRSFRRVVRSEQGSLRVLRPFDEVSRLLRGIRDYRVAVLEANP
RSFVVPSHTDAHCIGYVAEGEGVVTTIENGERRSYTIKQGHVFVAPAGAV
TYLANTDGRKKLVITKILHTISVPGEFQFFFGPGGRNPESFLSSFSKSIQ
RAAYKTSSDRLERLFGRHGQDKGIIVRATEEQTRELRRHASEGGHGPHWP
LPPFGESRGPYSLLDQRPSIANQHGQLYEADARSFHDLAEHDVSVSFANI
TAGSMSAPLYNTRSFKIAYVPNGKGYAEIVCPHRQSQGGESERERGKGRR
SEEEEESSEEQEEVGQGYHTIRARLSPGTAFVVPAGHPFVAVASRDSNLQ
IVCFEVHADRNEKVFLAGADNVLQKLDRVAKALSFASKAEEVDEVLGSRR
EKGFLPGPKESGGHEEREQEEEEREERHGGRGERERHGREEREKEEEERE
GRHGRGRREEVAETLLRMVTARM
Glb3
MAKIAAAAAAALCFAALVAVAVCQGEVERQRLRDLQCWQEVQESPLDACR
QVLDRQLTGGGGGGGVGPFRWGTGLRMRCCQQLQDVSRECRCAAIRSMVR
GYEEAMPPLEKGWWPWGRQQQPPPQGGGGGQGGYYYPCSRPGEGYGYGQG
GQRQMYPPCRPGTTGGGPRIGRVRLTKAREYAAGLPMMCRLSEPQECSIF
SGGDQY
Oat seed globulin
MATTRFPSLLFYSCIFLLCNGSMAQLFGQSFTPWQSSRQGGLRGCKFDRL
QAFEPLRQVRSQAGITEYFDEQNEQFRCAGVSVIRRVIEPQGLLLPQYHN
APGLVYILQGRGFTGLTFPGCPATFQQQFQQFDQARFAQGQSKSQNLKDE
HQRVHHIKQGDVVALPAGIVHWCYNDGDAPIVAVYVFDVNNNANQLEPRQ
KEFLLAGNNKREQQFGQNIFSGFSVQLLSEALGISQQAAQKIQSQNDQRG
EIIRVSQGLQFLKPFVSQQGPVEHQAYQPIQSQQEQSTQYQVGQSPQYQE
GQSTQYQSGQSWDQSFNGLEENFCSLEARQNIENPKRADTYNPRAGRITH
LNSKNFPTLNLVQMSATRVNLYQNAILSPYWNINAHSVMHMIQGRARVQV
VNNHGQTVFNDILRRGQLLIIPQHYVVLKKAEREGCQYISFKTTPNSMVS
YIAGKTSILRALPVDVLANAYRISRQESQNLKNNRGEEFGAFTPKFAQTG
SQSYQDEGESSSTEKASE
Sesame 11S globulin
MVAFKFLLALSLSLLVSAAIAQTREPRLTQGQQCRFQRISGAQPSLRIQS
EGGTTELWDERQEQFQCAGIVAMRSTIRPNGLSLPNYHPSPRLVYIERGQ
GLISIMVPGCAETYQVHRSQRTMERTEASEQQDRGSVRDLHQKVHRLRQG
DIVAIPSGAAHWCYNDGSEDLVAVSINDVNHLSNQLDQKFRAFYLAGGVP
RSGEQEQQARQTFHNIFRAFDAELLSEAFNVPQETIRRMQSEEEERGLIV
MARERMTFVRPDEEEGEQEHRGRQLDNGLEETFCTMKFRTNVESRREADI
FSRQAGRVHVVDRNKLPILKYMDLSAEKGNLYSNALVSPDWSMTGHTIVY
VTRGDAQVQVVDHNGQALMNDRVNQGEMFVVPQYYTSTARAGNNGFEWVA
FKTTGSPMRSPLAGYTSVIRAMPLQVITNSYQISPNQAQALKMNRGSQSF
LLSPGGRRS

### Example 7—Expression of Astaxanthin in Butyribacterium Methylotrophicum

Butyribacterium methylotrophicum do not natively produce astaxanthin but can produce lycopene, a key intermediate to astaxanthin. In order to enable B. methylotrophicum to produce astaxanthin from lycopene, three genes are needed: crtY, crtW, and crtZ.

A synthetic operon of these three genes is constructed with a constitutively active transcriptional promoter and then integrated into the chromosome of B. methylotrophicum or expressed from a replicating plasmid. Expression of these three genes allows astaxanthin to be produced.

Examples of the crtY, crtW, and crtZ genes are given.

crtY

crtY
ATGAACGGGCGCAGGGCAGATCTGGCGATTGTTGGCGGCGGCCTGTCGGG
CGGCCTGATCGCGCTGGCGCTGAGGAACGCGCGGCCCGAGCTCGACGTCA
GGCTGATAGAAGCGGGCGAGAGGCTGGGCGGCAATCACCGCTGGAGCTGG
TTCGAGAACGATCTCGGCAAAAGCGGCAACGAACTCATGCAGCCGTTTCG
CAAGACCGAATGGCAGGGCTACGATGTTTATTTCCCGAAATATTCCCGCC
GTCTGAAATCTCGCTACTATTCTCTGGCATCGCCCGATTTCGACGCCGGA
TTGCGGCGGGAATTGGCGCAAGACACTGTTCATACCGGCAGGAAAGTGGT
GGAATGCACGCCTGACAGTGTCACGCTGGAAGGGGGAGACGCCATACCGG
CCCGTGCCGTGGTGGATTGCCGAGGGTTCGAGCCGAGCGAGCTCGTGCGT
GGCGGCTGGCAGGTCTTCATGGGCCGCCACCTGCGCACCCATGCCCCGCA
CGGCATTACCCGACCGGTCATCATGGACGTGACCGTAGAACAGCTCGACG
GCTACCGCTTCGTTTACGTCCTCCCGCTCGGCGCAAGCGACATCTTCATC
GAGGACACCTATTACAATTCCAACCCCGAGCTGGATCGCGGCGCGCTGTC
GAGCCGGCTCGACCGATATTGCCGCCAGAACGGGTGGGAAGGCGACATCG
TCGGCAGCGAGACCGGGGTCCTGCCGGTCATTACCGGCGGCGATTTCGCT
GCCTATCGCCGCGAGCGCGAGCTCGACCGGATGCCGCAGGCGGGTGCCCG
CGCGCTGCTGGCCCACCCGCTGACCAGCTACACCCTGCCGCAGGCGGTCG
AAACCGCGCGCCTGATCGCGCGCAACGCGGACCTGCCTGGCGACCAGCTG
GCCGCGCTGCTCGCCGCCCATGCCCAGCGGCACTGGAATCGCACGAGCTA
TTACCGCCTATTGGGCAGGATACTTTTCGAAGCCCCGCAGCCTAAGGAAC
GCTATCGAATCTTCGAGCGTTTCTACACACTCGACGAGGACTTGATCGAG
CGGTTCTATGCCGCGCGTTCGCGCCCGCAGGAAAAGGCCCGCGTTTTGTG
GGGCAATCCGCCGGTCGCGATCCACCGCGCCATCGCGGCGATCTTCAGCA
AGAGTGCGCCGCTGGTGGTGCCGGACAGAACGAAAGGTCGCGTGTCGACA
TGA
crtW
ATGTCTGACGGTCCTGCCTTTTCGTTGCCTGCGCGCTCGCGTCAGCAGGC
GATCGGGTTGACGCTCGCCGTGCTGATCGCTGGCGCGTGGCTCGGCATCC
ACGCCTATGCGATGTTCGTTTTCGAGCTGAGTTGGCAGAACCTGCCCTTC
GCACTTCTGCTAGCAACGGTGCAAACGTGGCTGTCGGTTGGCGTCTTCAT
CGTTAGTCATGACGCAATGCACGGCTCGCTGGCACCCGGCAGGCCGACGC
TGAATTCGGCGATCGGCGCGTTTCTCTTGGCCCTTTACGCAGGCTTCGGC
TGGCGACGAATGCGTGACGCGCATTTTACCCACCACAAGCTGGCGGGCCA
TGCGGGAGACCCGGATTTCGACGAGCACAATCCGCGCAGTTTCTGGCGCT
GGTACGGAACCTTCTTCCGGCGCTATTTCAGCTGGCAATCGATCCTGTTC
GTGCATGTCGTTGTCGGCATCTACTGGCTAGTCCTCGACATTCCGATGGT
GCAGATCGTCCTGCTCTACGGCGCACCGGCACTGCTTTCATCGCTGCAGC
TTTTCTACTTCGGGACCTTTCGTCCACATCGGCGTTCCGAAGAGGGTTTT
GCCGACCGGCACAACGCGCGCAGCGATGGCTTCGGCACGTTGGCCAGCCT
CGCCACGTGCTTCCATTTCGGCTATCATCTCGAACATCATCGGCGTCCCG
ACGTGCCGTGGTGGGCCTTGCCCGGCGCGCGAGAAGCGGGGATCGGAATG
GAAGCGCAAAACGCATGA
crtZ
ATGAGCTGGTGGGCAATCGCCCTGATCGTATTCGGTGCCGTGGTCGGCAT
GGAGTTTTTTGCCTGGTTTGCCCACAAATACATCATGCACGGCTGGGGCT
GGTCCTGGCATCGCGACCATCATGAGCCGCACGACAACACACTGGAAAAG
AACGATCTTTTCGCGGTCGTGTTCGGATCCGTAGCGGCGCTGCTTTTTGT
TATAGGCGCCCTGTGGTCCGATCCTTTGTGGTGGGCTGCGGTCGGGATCA
CGCTTTACGGCGTGATTTATACGCTGGTGCACGACGGGCTGGTCCATCAG
CGCTACTGGCGCTGGACGCCCAAGCGCGGTTACGCGAAGCGGCTGGTCCA
GGCCCACCGGCTGCATCATGCCACGGTAGGCAAGGAGGGCGGGGTGAGTT
TCGGCTTCGTTTTCGCGCGCGATCCGGCAAAACTGAAGGCTGAACTCAAG
CAGCAGCGCGAACAGGGTCTCGCGGTCGTGCGCGACAGCATGGGCGCCTG
A

### Example 8—Expression of Omega-3 Fatty Acids in Butyribacterium Methylotrophicum

Butyribacterium methylotrophicum do not natively produce omega-3 fatty acids but can produce oleic acid from its native fatty acid biosynthesis. Eicosapentaenoic acid (EPA), an important omega-3 fatty acid, can be produced from oleic acid with expression of four genes pfaABCD, and then docosahexaenoic acid (DHA), another important omega-3 fatty acid, can be produced from EPA with an additional gene pfaE. In order to enable B. methylotrophicum to produce omega-3 fatty acids, these five genes are needed.

A synthetic operon of these five genes is constructed with a constitutively active transcriptional promoter and then integrated into the chromosome of B. methylotrophicum or expressed from a replicating plasmid. Expression of these five genes allows EPA and DHA to be produced.

Examples of the pfaA, pfaB, pfaC, pfaD, and pfaE genes are given.

pfaA
ATGAGCCAGACCTCTAAACCTACAAACTCAGCAACTGAGCAAGCACAAGA
CTCACAAGCTGACTCTCGTTTAAATAAACGACTAAAAGATATGCCAATTG
CTATTGTTGGCATGGCGAGTATTTTTGCAAACTCTCGCTATTTGAATAAG
TTTTGGGACTTAATCAGCGAAAAAATTGATGCGATTACTGAATTACCATC
AACTCACTGGCAGCCTGAAGAATATTACGACGCAGATAAAACCGCAGCAG
ACAAAAGCTACTGTAAACGTGGTGGCTTTTTGCCAGATGTAGACTTCAAC
CCAATGGAGTTTGGCCTGCCGCCAAACATTTTGGAACTGACCGATTCATC
GCAACTATTATCACTCATCGTTGCTAAAGAAGTGTTGGCTGATGCTAACT
TACCTGAGAATTACGACCGCGATAAAATTGGTATCACCTTAGGTGTCGGC
GGTGGTCAAAAAATTAGCCACAGCCTAACAGCGCGTCTGCAATACCCAGT
ATTGAAGAAAGTATTCGCCAATAGCGGCATTAGTGACACCGACAGCGAAA
TGCTTATCAAGAAATTCCAAGACCAATATGTACACTGGGAAGAAAACTCG
TTCCCAGGTTCACTTGGTAACGTTATTGCGGGCCGTATCGCCAACCGCTT
CGATTTTGGCGGCATGAACTGTGTGGTTGATGCTGCCTGTGCTGGATCAC
TTGCTGCTATGCGTATGGCGCTAACAGAGCTAACTGAAGGTCGCTCTGAA
ATGATGATCACCGGTGGTGTGTGTACTGATAACTCACCCTCTATGTATAT
GAGCTTTTCAAAAACGCCCGCCTTTACCACTAACGAAACCATTCAGCCAT
TTGATATCGACTCAAAAGGCATGATGATTGGTGAAGGTATTGGCATGGTG
GCGCTAAAGCGTCTTGAAGATGCAGAGCGCGATGGCGACCGCATTTACTC
TGTAATTAAAGGTGTGGGTGCATCATCTGACGGTAAGTTTAAATCAATCT
ATGCCCCTCGCCCATCAGGCCAAGCTAAAGCACTTAACCGTGCCTATGAT
GACGCAGGTTTTGCGCCGCATACCTTAGGTCTAATTGAAGCTCACGGAAC
AGGTACTGCAGCAGGTGACGCGGCAGAGTTTGCCGGCCTTTGCTCAGTAT
TTGCTGAAGGCAACGATACCAAGCAACACATTGCGCTAGGTTCAGTTAAA
TCACAAATTGGTCATACTAAATCAACTGCAGGTACAGCAGGTTTAATTAA
AGCTGCTCTTGCTTTGCATCACAAGGTACTGCCGCCGACCATTAACGTTA
GTCAGCCAAGCCCTAAACTTGATATCGAAAACTCACCGTTTTATCTAAAC
ACTGAGACTCGTCCATGGTTACCACGTGTTGATGGTACGCCGCGCCGCGC
GGGTATTAGCTCATTTGGTTTTGGTGGCACTAACTTCCATTTTGTACTAG
AAGAGTACAACCAAGAACACAGCCGTACTGATAGCGAAAAAGCTAAGTAT
CGTCAACGCCAAGTGGCGCAAAGCTTCCTTGTTAGCGCAAGCGATAAAGC
ATCGCTAATTAACGAGTTAAACGTACTAGCAGCATCTGCAAGCCAAGCTG
AGTTTATCCTCAAAGATGCAGCAGCAAACTATGGCGTACGTGAGCTTGAT
AAAAATGCACCACGGATCGGTTTAGTTGCAAACACAGCTGAAGAGTTAGC
AGGCCTAATTAAGCAAGCACTTGCCAAACTAGCAGCTAGCGATGATAACG
CATGGCAGCTACCTGGTGGCACTAGCTACCGCGCCGCTGCAGTAGAAGGT
AAAGTTGCCGCACTGTTTGCTGGCCAAGGTTCACAATATCTCAATATGGG
CCGTGACCTTACTTGTTATTACCCAGAGATGCGTCAGCAATTTGTAACTG
CAGATAAAGTATTTGCCGCAAATGATAAAACGCCGTTATCGCAAACTCTG
TATCCAAAGCCTGTATTTAATAAAGATGAATTAAAGGCTCAAGAAGCCAT
TTTGACCAATACCGCCAATGCCCAAAGCGCAATTGGTGCGATTTCAATGG
GTCAATACGATTTGTTTACTGCGGCTGGCTTTAATGCCGACATGGTTGCA
GGCCATAGCTTTGGTGAGCTAAGTGCACTGTGTGCTGCAGGTGTTATTTC
AGCTGATGACTACTACAAGCTGGCTTTTGCTCGTGGTGAGGCTATGGCAA
CAAAAGCACCGGCTAAAGACGGCGTTGAAGCAGATGCAGGAGCAATGTTT
GCAATCATAACCAAGAGTGCTGCAGACCTTGAAACCGTTGAAGCCACCAT
CGCTAAATTTGATGGGGTGAAAGTCGCTAACTATAACGCGCCAACGCAAT
CAGTAATTGCAGGCCCAACAGCAACTACCGCTGATGCGGCTAAAGCGCTA
ACTGAGCTTGGTTACAAAGCGATTAACCTGCCAGTATCAGGTGCATTCCA
CACTGAACTTGTTGGTCACGCTCAAGCGCCATTTGCTAAAGCGATTGACG
CAGCCAAATTTACTAAAACAAGCCGAGCACTTTACTCAAATGCAACTGGC
GGACTTTATGAAAGCACTGCTGCAAAGATTAAAGCCTCGTTTAAGAAACA
TATGCTTCAATCAGTGCGCTTTACTAGCCAGCTAGAAGCCATGTACAACG
ACGGCGCCCGTGTATTTGTTGAATTTGGTCCAAAGAACATCTTACAAAAA
TTAGTTCAAGGCACGCTTGTCAACACTGAAAATGAAGTTTGCACTATCTC
TATCAACCCTAATCCTAAAGTTGATAGTGATCTGCAGCTTAAGCAAGCAG
CAATGCAGCTAGCGGTTACTGGTGTGGTACTCAGTGAAATTGACCCATAC
CAAGCCGATATTGCCGCACCAGCGAAAAAGTCGCCAATGAGCATTTCGCT
TAATGCTGCTAACCATATCAGCAAAGCAACTCGCGCTAAGATGGCCAAGT
CTTTAGAGACAGGTATCGTCACCTCGCAAATAGAACATGTTATTGAAGAA
AAAATCGTTGAAGTTGAGAAACTGGTTGAAGTCGAAAAGATCGTCGAAAA
AGTGGTTGAAGTAGAGAAAGTTGTTGAGGTTGAAGCTCCTGTTAATTCAG
TGCAAGCCAATGCAATTCAAACCCGTTCAGTTGTCGCTCCAGTAATAGAG
AACCAAGTCGTGTCTAAAAACAGTAAGCCAGCAGTCCAGAGCATTAGTGG
TGATGCACTCAGCAACTTTTTTGCTGCACAGCAGCAAACCGCACAGTTGC
ATCAGCAGTTCTTAGCTATTCCGCAGCAATATGGTGAGACGTTCACTACG
CTGATGACCGAGCAAGCTAAACTGGCAAGTTCTGGTGTTGCAATTCCAGA
GAGTCTGCAACGCTCAATGGAGCAATTCCACCAACTACAAGCGCAAACAC
TACAAAGCCACACCCAGTTCCTTGAGATGCAAGCGGGTAGCAACATTGCA
GCGTTAAACCTACTCAATAGCAGCCAAGCAACTTACGCTCCAGCCATTCA
CAATGAAGCGATTCAAAGCCAAGTGGTTCAAAGCCAAACTGCAGTCCAGC
CAGTAATTTCAACACAAGTTAACCATGTGTCAGAGCAGCCAACTCAAGCT
CCAGCTCCAAAAGCGCAGCCAGCACCTGTGACAACTGCAGTTCAAACTGC
TCCGGCACAAGTTGTTCGTCAAGCCGCACCAGTTCAAGCCGCTATTGAAC
CGATTAATACAAGTGTTGCGACTACAACGCCTTCAGCCTTCAGCGCCGAA
ACAGCCCTGAGCGCAACAAAAGTCCAAGCCACTATGCTTGAAGTGGTTGC
TGAGAAAACCGGTTACCCAACTGAAATGCTAGAGCTTGAAATGGATATGG
AAGCCGATTTAGGCATCGATTCTATCAAGCGTGTAGAAATTCTTGGCACA
GTACAAGATGAGCTACCGGGTCTACCTGAGCTTAGCCCTGAAGATCTAGC
TGAGTGTCGAACGCTAGGCGAAATCGTTGACTATATGGGCAGTAAACTGC
CGGCTGAAGGCTCTATGAATTCTCAGCTGTCTACAGGTTCCGCAGCTGCG
ACTCCTGCAGCGAATGGTCTTTCTGCGGAGAAAGTTCAAGCGACTATGAT
GTCTGTGGTTGCCGAAAAGACTGGCTACCCAACTGAAATGCTAGAGCTTG
AAATGGATATGGAAGCCGATTTAGGCATAGATTCTATCAAGCGCGTTGAA
ATTCTTGGCACAGTACAAGATGAGCTACCGGGTCTACCTGAGCTTAGCCC
TGAAGATCTAGCTGAGTGTCGTACTCTAGGCGAAATCGTTGACTATATGA
ACTCTAAACTCGCTGACGGCTCTAAGCTGCCGGCTGAAGGCTCTATGAAT
TCTCAGCTGTCTACAAGTGCCGCAGCTGCGACTCCTGCAGCGAATGGTCT
CTCTGCGGAGAAAGTTCAAGCGACTATGATGTCTGTGGTTGCCGAAAAGA
CTGGCTACCCAACTGAAATGCTAGAACTTGAAATGGATATGGAAGCTGAC
CTTGGCATCGATTCAATCAAGCGCGTTGAAATTCTTGGCACAGTACAAGA
TGAGCTACCGGGTTTACCTGAGCTAAATCCAGAAGATTTGGCAGAGTGTC
GTACTCTTGGCGAAATCGTGACTTATATGAACTCTAAACTCGCTGACGGC
TCTAAGCTGCCAGCTGAAGGCTCTATGCACTATCAGCTGTCTACAAGTAC
CGCTGCTGCGACTCCTGTAGCGAATGGTCTCTCTGCAGAAAAAGTTCAAG
CGACCATGATGTCTGTAGTTGCAGATAAAACTGGCTACCCAACTGAAATG
CTTGAACTTGAAATGGATATGGAAGCCGATTTAGGTATCGATTCTATCAA
GCGCGTTGAAATTCTTGGCACAGTACAAGATGAGCTACCGGGTTTACCTG
AGCTAAATCCAGAAGATCTAGCAGAGTGTCGCACCCTAGGCGAAATCGTT
GACTATATGGGCAGTAAACTGCCGGCTGAAGGCTCTGCTAATACAAGTGC
CGCTGCGTCTCTTAATGTTAGTGCCGTTGCGGCGCCTCAAGCTGCTGCGA
CTCCTGTATCGAACGGTCTCTCTGCAGAGAAAGTGCAAAGCACTATGATG
TCAGTAGTTGCAGAAAAGACCGGCTACCCAACTGAAATGCTAGAACTTGG
CATGGATATGGAAGCCGATTTAGGTATCGACTCAATTAAACGCGTTGAGA
TTCTTGGCACAGTACAAGATGAGCTACCGGGTCTACCAGAGCTTAATCCT
GAAGATTTAGCTGAGTGCCGTACGCTGGGCGAAATCGTTGACTATATGAA
CTCTAAGCTGGCTGACGGCTCTAAGCTTCCAGCTGAAGGCTCTGCTAATA
CAAGTGCCACTGCTGCGACTCCTGCAGTGAATGGTCTTTCTGCTGACAAG
GTACAGGCGACTATGATGTCTGTAGTTGCTGAAAAGACCGGCTACCCAAC
TGAAATGCTAGAACTTGGCATGGATATGGAAGCAGACCTTGGTATTGATT
CTATTAAGCGCGTTGAAATTCTTGGCACAGTACAAGATGAGCTCCCAGGT
TTACCTGAGCTTAATCCTGAAGATCTCGCTGAGTGCCGCACGCTTGGCGA
AATCGTTAGCTATATGAACTCTCAACTGGCTGATGGCTCTAAACTTTCTA
CAAGTGCGGCTGAAGGCTCTGCTGATACAAGTGCTGCAAATGCTGCAAAG
CCGGCAGCAATTTCGGCAGAACCAAGTGTTGAGCTTCCTCCTCATAGCGA
GGTAGCGCTAAAAAAGCTTAATGCGGCGAACAAGCTAGAAAATTGTTTCG
CCGCAGACGCAAGTGTTGTGATTAACGATGATGGTCACAACGCAGGCGTT
TTAGCTGAGAAACTTATTAAACAAGGCCTAAAAGTAGCCGTTGTGCGTTT
ACCGAAAGGTCAGCCTCAATCGCCACTTTCAAGCGATGTTGCTAGCTTTG
AGCTTGCCTCAAGCCAAGAATCTGAGCTTGAAGCCAGTATCACTGCAGTT
ATCGCGCAGATTGAAACTCAGGTTGGCGCTATTGGTGGCTTTATTCACTT
GCAACCAGAAGCGAATACAGAAGAGCAAACGGCAGTAAACCTAGATGCGC
AAAGTTTTACTCACGTTAGCAATGCGTTCTTGTGGGCCAAATTATTGCAA
CCAAAGCTCGTTGCTGGAGCAGATGCGCGTCGCTGTTTTGTAACAGTAAG
CCGTATCGACGGTGGCTTTGGTTACCTAAATACTGACGCCCTAAAAGATG
CTGAGCTAAACCAAGCAGCATTAGCTGGTTTAACTAAAACCTTAAGCCAT
GAATGGCCACAAGTGTTCTGTCGCGCGCTAGATATTGCAACAGATGTTGA
TGCAACCCATCTTGCTGATGCAATCACCAGTGAACTATTTGATAGCCAAG
CTCAGCTACCTGAAGTGGGCTTAAGCTTAATTGATGGCAAAGTTAACCGC
GTAACTCTAGTTGCTGCTGAAGCTGCAGATAAAACAGCAAAAGCAGAGCT
TAACAGCACAGATAAAATCTTAGTGACTGGTGGGGCAAAAGGGGTGACAT
TTGAATGTGCACTGGCATTAGCATCTCGCAGCCAGTCTCACTTTATCTTA
GCTGGGCGCAGTGAATTACAAGCTTTACCAAGCTGGGCTGAGGGTAAGCA
AACTAGCGAGCTAAAATCAGCTGCAATCGCACATATTATTTCTACTGGTC
AAAAGCCAACGCCTAAGCAAGTTGAAGCCGCTGTGTGGCCAGTGCAAAGC
AGCATTGAAATTAATGCCGCCCTAGCCGCCTTTAACAAAGTTGGCGCCTC
AGCTGAATACGTCAGCATGGATGTTACCGATAGCGCCGCAATCACAGCAG
CACTTAATGGTCGCTCAAATGAGATCACCGGTCTTATTCATGGCGCAGGT
GTACTAGCCGACAAGCATATTCAAGACAAGACTCTTGCTGAACTTGCTAA
AGTTTATGGCACTAAAGTCAACGGCCTAAAAGCGCTGCTCGCGGCACTTG
AGCCAAGCAAAATTAAATTACTTGCTATGTTCTCATCTGCAGCAGGTTTT
TACGGTAATATCGGCCAAAGCGATTACGCGATGTCGAACGATATTCTTAA
CAAGGCAGCGCTGCAGTTCACCGCTCGCAACCCACAAGCTAAAGTCATGA
GCTTTAACTGGGGTCCTTGGGATGGCGGCATGGTTAACCCAGCGCTTAAA
AAGATGTTTACCGAGCGTGGTGTGTACGTTATTCCACTAAAAGCAGGTGC
AGAGCTATTTGCCACTCAGCTATTGGCTGAAACTGGCGTGCAGTTGCTCA
TTGGTACGTCAATGCAAGGTGGCAGCGACACTAAAGCAACTGAGACTGCT
TCTGTAAAAAAGCTTAATGCGGGTGAGGTGCTAAGTGCATCGCATCCGCG
TGCTGGTGCACAAAAAACACCACTACAAGCTGTCACTGCAACGCGTCTGT
TAACCCCAAGTGCCATGGTCTTCATTGAAGATCACCGCATTGGCGGTAAC
AGTGTGTTGCCAACGGTATGCGCCATCGACTGGATGCGTGAAGCGGCAAG
CGACATGCTTGGCGCTCAAGTTAAGGTACTTGATTACAAGCTATTAAAAG
GCATTGTATTTGAGACTGATGAGCCGCAAGAGTTAACACTTGAGCTAACG
CCAGACGATTCAGACGAAGCTACGCTACAAGCATTAATCAGCTGTAATGG
GCGTCCGCAATACAAGGCGACGCTTATCAGTGATAATGCCGATATTAAGC
AACTTAACAAGCAGTTTGATTTAAGCGCTAAGGCGATTACCACAGCAAAA
GAGCTTTATAGCAACGGCACCTTGTTCCACGGTCCGCGTCTACAAGGGAT
CCAATCTGTAGTGCAGTTCGATGATCAAGGCTTAATTGCTAAAGTCGCTC
TGCCTAAGGTTGAACTTAGCGATTGTGGTGAGTTCTTGCCGCAAACCCAC
ATGGGTGGCAGTCAACCTTTTGCTGAGGACTTGCTATTACAAGCTATGCT
GGTTTGGGCTCGCCTTAAAACTGGCTCGGCAAGTTTGCCATCAAGCATTG
GTGAGTTTACCTCATACCAACCAATGGCCTTTGGTGAAACTGGTACCATA
GAGCTTGAAGTGATTAAGCACAACAAACGCTCACTTGAAGCGAATGTTGC
GCTATATCGTGACAACGGCGAGTTAAGTGCCATGTTTAAGTCAGCTAAAA
TCACCATTAGCAAAAGCTTAAATTCAGCATTTTTACCTGCTGTCTTAGCA
AACGACAGTGAGGCGAATTAG
pfaB
GTGGAACAAACGCCTAAAGCTAGTGCGATGCCGCTGCGCATCGCACTTAT
CTTACTGCCAACACCGCAGTTTGAAGTTAACTCTGTCGACCAGTCAGTAT
TAGCCAGCTATCAAACACTGCAGCCTGAGCTAAATGCCCTGCTTAATAGT
GCGCCGACACCTGAAATGCTCAGCATCACTATCTCAGATGATAGCGATGC
AAACAGCTTTGAGTCGCAGCTAAATGCTGCGACCAACGCAATTAACAATG
GCTATATCGTCAAGCTTGCTACGGCAACTCACGCTTTGTTAATGCTGCCT
GCATTAAAAGCGGCGCAAATGCGGATCCATCCTCATGCGCAGCTTGCCGC
TATGCAGCAAGCTAAATCGACGCCAATGAGTCAAGTATCTGGTGAGCTAA
AGCTTGGCGCTAATGCGCTAAGCCTAGCTCAGACTAATGCGCTGTCTCAT
GCTTTAAGCCAAGCCAAGCGTAACTTAACTGATGTCAGCGTGAATGAGTG
TTTTGAGAACCTCAAAAGTGAACAGCAGTTCACAGAGGTTTATTCGCTTA
TTCAGCAACTTGCTAGCCGCACCCATGTGAGAAAAGAGGTTAATCAAGGT
GTGGAACTTGGCCCTAAACAAGCCAAAAGCCACTATTGGTTTAGCGAATT
TCACCAAAACCGTGTTGCTGCCATCAACTTTATTAATGGCCAACAAGCAA
CCAGCTATGTGCTTACTCAAGGTTCAGGATTGTTAGCTGCGAAATCAATG
CTAAACCAGCAAAGATTAATGTTTATCTTGCCGGGTAACAGTCAGCAACA
AATAACCGCATCAATAACTCAGTTAATGCAGCAATTAGAGCGTTTGCAGG
TAACTGAGGTTAATGAGCTTTCTCTAGAATGCCAACTAGAGCTGCTCAGC
ATAATGTATGACAACTTAGTCAACGCAGACAAACTCACTACTCGCGATAG
TAAGCCCGCTTATCAGGCTGTGATTCAAGCAAGCTCTGTTAGCGCTGCAA
AGCAAGAGTTAAGCGCGCTTAACGATGCACTCACAGCGCTGTTTGCTGAG
CAAACAAACGCCACATCAACGAATAAAGGCTTAATCCAATACAAAACACC
GGCGGGCAGTTACTTAACCCTAACACCGCTTGGCAGCAACAATGACAACG
CCCAAGCGGGTCTTGCTTTTGTCTATCCGGGTGTGGGAACGGTTTACGCC
GATATGCTTAATGAGCTGCATCAGTACTTCCCTGCGCTTTACGCCAAACT
TGAGCGTGAAGGCGATTTAAAGGCGATGCTACAAGCAGAAGATATCTATC
ATCTTGACCCTAAACATGCTGCCCAAATGAGCTTAGGTGACTTAGCCATT
GCTGGCGTGGGGAGCAGCTACCTGTTAACTCAGCTGCTCACCGATGAGTT
TAATATTAAGCCTAATTTTGCATTAGGTTACTCAATGGGTGAAGCATCAA
TGTGGGCAAGCTTAGGCGTATGGCAAAACCCGCATGCGCTGATCAGCAAA
ACCCAAACCGACCCGCTATTTACTTCTGCTATTTCCGGCAAATTGACCGC
GGTTAGACAAGCTTGGCAGCTTGATGATACCGCAGCGGAAATCCAGTGGA
ATAGCTTTGTGGTTAGAAGTGAAGCAGCGCCGATTGAAGCCTTGCTAAAA
GATTACCCACACGCTTACCTCGCGATTATTCAAGGGGATACCTGCGTAAT
CGCTGGCTGTGAAATCCAATGTAAAGCGCTACTTGCAGCACTGGGTAAAC
GCGGTATTGCAGCTAATCGTGTAACGGCGATGCATACGCAGCCTGCGATG
CAAGAGCATCAAAATGTGATGGATTTTTATCTGCAACCGTTAAAAGCAGA
GCTTCCTAGTGAAATAAGCTTTATCAGCGCCGCTGATTTAACTGCCAAGC
AAACGGTGAGTGAGCAAGCACTTAGCAGCCAAGTCGTTGCTCAGTCTATT
GCCGACACCTTCTGCCAAACCTTGGACTTTACCGCGCTAGTACATCACGC
CCAACATCAAGGCGCTAAGCTGTTTGTTGAAATTGGCGCGGATAGACAAA
ACTGCACCTTGATAGACAAGATTGTTAAACAAGATGGTGCCAGCAGTGTA
CAACATCAACCTTGTTGCACAGTGCCTATGAACGCAAAAGGTAGCCAAGA
TATTACCAGCGTGATTAAAGCGCTTGGCCAATTAATTAGCCATCAGGTGC
CATTATCGGTGCAACCATTTATTGATGGACTCAAGCGCGAGCTAACACTT
TGCCAATTGACCAGCCAACAGCTGGCAGCACATGCAAATGTTGACAGCAA
GTTTGAGTCTAACCAAGACCATTTACTTCAAGGGGAAGTCTAA
pfaC
ATGTCATTACCAGACAATGCTTCTAACCACCTTTCTGCCAACCAGAAAGG
CGCATCTCAGGCAAGTAAAACCAGTAAGCAAAGCAAAATCGCCATTGTCG
GTTTAGCCACTCTGTATCCAGACGCTAAAACCCCGCAAGAATTTTGGCAG
AATTTGCTGGATAAACGCGACTCTCGCAGCACCTTAACTAACGAAAAACT
CGGCGCTAACAGCCAAGATTATCAAGGTGTGCAAGGCCAATCTGACCGTT
TTTATTGTAATAAAGGCGGCTACATTGAGAACTTCAGCTTTAATGCTGCA
GGCTACAAATTGCCGGAGCAAAGCTTAAATGGCTTGGACGACAGCTTCCT
TTGGGCGCTCGATACTAGCCGTAACGCACTAATTGATGCTGGTATTGATA
TCAACGGCGCTGATTTAAGCCGCGCAGGTGTAGTCATGGGCGCGCTGTCG
TTCCCAACTACCCGCTCAAACGATCTGTTTTTGCCAATTTATCACAGCGC
CGTTGAAAAAGCCCTGCAAGATAAACTAGGCGTAAAGGCATTTAAGCTAA
GCCCAACTAATGCTCATACCGCTCGCGCGGCAAATGAGAGCAGCCTAAAT
GCAGCCAATGGTGCCATTGCCCATAACAGCTCAAAAGTGGTGGCCGATGC
ACTTGGCCTTGGCGGCGCACAACTAAGCCTAGATGCTGCCTGTGCTAGTT
CGGTTTACTCATTAAAGCTTGCCTGCGATTACCTAAGCACTGGCAAAGCC
GATATCATGCTAGCAGGCGCAGTATCTGGCGCGGATCCTTTCTTTATTAA
TATGGGATTCTCAATCTTCCACGCCTACCCAGACCATGGTATCTCAGTAC
CGTTTGATGCCAGCAGTAAAGGTTTGTTTGCTGGCGAAGGCGCTGGCGTA
TTAGTGCTTAAACGTCTTGAAGATGCCGAGCGCGACAATGACAAAATCTA
TGCGGTTGTTAGCGGCGTAGGTCTATCAAACGACGGTAAAGGCCAGTTTG
TATTAAGCCCTAATCCAAAAGGTCAGGTGAAGGCCTTTGAACGTGCTTAT
GCTGCCAGTGACATTGAGCCAAAAGACATTGAAGTGATTGAGTGCCACGC
AACAGGCACACCGCTTGGCGATAAAATTGAGCTCACTTCAATGGAAACCT
TCTTTGAAGACAAGCTGCAAGGCACCGATGCACCGTTAATTGGCTCAGCT
AAGTCTAACTTAGGCCACCTATTAACTGCAGCGCATGCGGGGATCATGAA
GATGATCTTCGCCATGAAAGAAGGTTACCTGCCGCCAAGTATCAATATTA
GTGATGCTATCGCTTCGCCGAAAAAACTCTTCGGTAAACCAACCCTGCCT
AGCATGGTTCAAGGCTGGCCAGATAAGCCATCGAATAATCATTTTGGTGT
AAGAACCCGTCACGCAGGCGTATCGGTATTTGGCTTTGGTGGCTGTAACG
CCCATCTGTTGCTTGAGTCATACAACGGCAAAGGAACAGTAAAGGCAGAA
GCCACTCAAGTACCGCGTCAAGCTGAGCCGCTAAAAGTGGTTGGCCTTGC
CTCGCACTTTGGGCCTCTTAGCAGCATTAATGCACTCAACAATGCTGTGA
CCCAAGATGGGAATGGCTTTATCGAACTGCCGAAAAAGCGCTGGAAAGGC
CTTGAAAAGCACAGTGAACTGTTAGCTGAATTTGGCTTAGCATCTGCGCC
AAAAGGTGCTTATGTTGATAACTTCGAGCTGGACTTTTTACGCTTTAAAC
TGCCGCCAAACGAAGATGACCGTTTGATCTCACAGCAGCTAATGCTAATG
CGAGTAACAGACGAAGCCATTCGTGATGCCAAGCTTGAGCCGGGGCAAAA
AGTAGCTGTATTAGTGGCAATGGAAACTGAGCTTGAACTGCATCAGTTCC
GCGGCCGGGTTAACTTGCATACTCAATTAGCGCAAAGTCTTGCCGCCATG
GGCGTGAGTTTATCAACGGATGAATACCAAGCGCTTGAAGCCATCGCCAT
GGACAGCGTGCTTGATGCTGCCAAGCTCAATCAGTACACCAGCTTTATTG
GTAATATTATGGCGTCACGCGTGGCGTCACTATGGGACTTTAATGGCCCA
GCCTTCACTATTTCAGCAGCAGAGCAATCTGTGAGCCGCTGTATCGATGT
GGCGCAAAACCTCATCATGGAGGATAACCTAGATGCGGTGGTGATTGCAG
CGGTCGATCTCTCTGGTAGCTTTGAGCAAGTCATTCTTAAAAATGCCATT
GCACCTGTAGCCATTGAGCCAAACCTCGAAGCAAGCCTTAATCCAACATC
AGCAAGCTGGAATGTCGGTGAAGGTGCTGGCGCGGTCGTGCTTGTTAAAA
ATGAAGCTACATCGGGCTGCTCATACGGCCAAATTGATGCACTTGGCTTT
GCTAAAACTGCCGAAACAGCGTTGGCTACCGACAAGCTACTGAGCCAAAC
TGCCACAGACTTTAATAAGGTTAAAGTGATTGAAACTATGGCAGCGCCTG
CTAGCCAAATTCAATTAGCGCCAATAGTTAGCTCTCAAGTGACTCACACT
GCTGCAGAGCAGCGTGTTGGTCACTGCTTTGCTGCAGCGGGTATGGCAAG
CCTATTACACGGCTTACTTAACTTAAATACTGTAGCCCAAACCAATAAAG
CCAATTGCGCGCTTATCAACAATATCAGTGAAAACCAATTATCACAGCTG
TTGATTAGCCAAACAGCGAGCGAACAACAAGCATTAACCGCGCGTTTAAG
CAATGAGCTTAAATCCGATGCTAAACACCAACTGGTTAAGCAAGTCACCT
TAGGTGGCCGTGATATCTACCAGCATATTGTTGATACACCGCTTGCAAGC
CTTGAAAGCATTACTCAGAAATTGGCGCAAGCGACAGCATCGACAGTGGT
CAACCAAGTTAAACCTATTAAGGCCGCTGGCTCAGTCGAAATGGCTAACT
CATTCGAAACGGAAAGCTCAGCAGAGCCACAAATAACAATTGCAGCACAA
CAGACTGCAAACATTGGCGTCACCGCTCAGGCAACCAAACGTGAATTAGG
TACCCCACCAATGACAACAAATACCATTGCTAATACAGCAAATAATTTAG
ACAAGACTCTTGAGACTGTTGCTGGCAATACTGTTGCTAGCAAGGTTGGC
TCTGGCGACATAGTCAATTTTCAACAGAACCAACAATTGGCTCAACAAGC
TCACCTCGCCTTTCTTGAAAGCCGCAGTGCGGGTATGAAGGTGGCTGATG
CTTTATTGAAGCAACAGCTAGCTCAAGTAACAGGCCAAACTATCGATAAT
CAGGCCCTCGATACTCAAGCCGTCGATACTCAAACAAGCGAGAATGTAGC
GATTGCCGCAGAATCACCAGTTCAAGTTACAACACCTGTTCAAGTTACAA
CACCTGTTCAAATCAGTGTTGTGGAGTTAAAACCAGATCACGCTAATGTG
CCACCATACACGCCGCCAGTGCCTGCATTAAAGCCGTGTATCTGGAACTA
TGCCGATTTAGTTGAGTACGCAGAAGGCGATATCGCCAAGGTATTTGGCA
GTGATTATGCCATTATCGACAGCTACTCGCGCCGCGTACGTCTACCGACC
ACTGACTACCTGTTGGTATCGCGCGTGACCAAACTTGATGCGACCATCAA
TCAATTTAAGCCATGCTCAATGACCACTGAGTACGACATCCCTGTTGATG
CGCCGTACTTAGTAGACGGACAAATCCCTTGGGCGGTAGCAGTAGAATCA
GGCCAATGTGACTTGATGCTTATTAGCTATCTCGGTATCGACTTTGAGAA
CAAAGGCGAGCGGGTTTATCGACTACTCGATTGTACCCTCACCTTCCTAG
GCGACTTGCCACGTGGCGGAGATACCCTACGTTACGACATTAAGATCAAT
AACTATGCTCGCAACGGCGACACCCTGCTGTTCTTCTTCTCGTATGAGTG
TTTTGTTGGCGACAAGATGATCCTCAAGATGGATGGCGGCTGCGCTGGCT
TCTTCACTGATGAAGAGCTTGCCGACGGTAAAGGCGTGATTCGCACAGAA
GAAGAGATTAAAGCTCGCAGCCTAGTGCAAAAGCAACGCTTTAATCCGTT
ACTAGATTGTCCTAAAACCCAATTTAGTTATGGTGATATTCATAAGCTAT
TAACTGCTGATATTGAGGGTTGTTTTGGCCCAAGCCACAGTGGCGTCCAC
CAGCCGTCACTTTGTTTCGCATCTGAAAAATTCTTGATGATTGAACAAGT
CAGCAAGGTTGATCGCACTGGCGGTACTTGGGGACTTGGCTTAATTGAGG
GTCATAAGCAGCTTGAAGCAGACCACTGGTACTTCCCATGTCATTTCAAG
GGCGACCAAGTGATGGCTGGCTCGCTAATGGCTGAAGGTTGTGGCCAGTT
ATTGCAGTTCTATATGCTGCACCTTGGTATGCATACCCAAACTAAAAATG
GTCGTTTCCAACCTCTTGAAAACGCCTCACAGCAAGTACGCTGTCGCGGT
CAAGTGCTGCCACAATCAGGCGTGCTAACTTACCGTATGGAAGTGACTGA
AATCGGTTTCAGTCCACGCCCATATGCTAAAGCTAACATCGATATCTTGC
TTAATGGCAAAGCGGTAGTGGATTTCCAAAACCTAGGGGTGATGATAAAA
GAGGAAGATGAGTGTACTCGTTATCCACTTTTGACTGAATCAACAACGGC
TAGCACTGCACAAGTAAACGCTCAAACAAGTGCGAAAAAGGTATACAAGC
CAGCATCAGTCAATGCGCCATTAATGGCACAAATTCCTGATCTGACTAAA
GAGCCAAACAAGGGCGTTATTCCGATTTCCCATGTTGAAGCACCAATTAC
GCCAGACTACCCGAACCGTGTACCTGATACAGTGCCATTCACGCCGTATC
ACATGTTTGAGTTTGCTACAGGCAATATCGAAAACTGTTTCGGGCCAGAG
TTCTCAATCTATCGCGGCATGATCCCACCACGTACACCATGCGGTGACTT
ACAAGTGACCACACGTGTGATTGAAGTTAACGGTAAGCGTGGCGACTTTA
AAAAGCCATCATCGTGTATCGCTGAATATGAAGTGCCTGCAGATGCGTGG
TATTTCGATAAAAACAGCCACGGCGCAGTGATGCCATATTCAATTTTAAT
GGAGATCTCACTGCAACCTAACGGCTTTATCTCAGGTTACATGGGCACAA
CCCTAGGCTTCCCTGGCCTTGAGCTGTTCTTCCGTAACTTAGACGGTAGC
GGTGAGTTACTACGTGAAGTAGATTTACGTGGTAAAACCATCCGTAACGA
CTCACGTTTATTATCAACAGTGATGGCCGGCACTAACATCATCCAAAGCT
TTAGCTTCGAGCTAAGCACTGACGGTGAGCCTTTCTATCGCGGCACTGCG
GTATTTGGCTATTTTAAAGGTGACGCACTTAAAGATCAGCTAGGCCTAGA
TAACGGTAAAGTCACTCAGCCATGGCATGTAGCTAACGGCGTTGCTGCAA
GCACTAAGGTGAACCTGCTTGATAAGAGCTGCCGTCACTTTAATGCGCCA
GCTAACCAGCCACACTATCGTCTAGCCGGTGGTCAGCTGAACTTTATCGA
CAGTGTTGAAATTGTTGATAATGGCGGCACCGAAGGTTTAGGTTACTTGT
ATGCCGAGCGCACCATTGACCCAAGTGATTGGTTCTTCCAGTTCCACTTC
CACCAAGATCCGGTTATGCCAGGCTCCTTAGGTGTTGAAGCAATTATTGA
AACCATGCAAGCTTACGCTATTAGTAAAGACTTGGGCGCAGATTTCAAAA
ATCCTAAGTTTGGTCAGATTTTATCGAACATCAAGTGGAAGTATCGCGGT
CAAATCAATCCGCTGAACAAGCAGATGTCTATGGATGTCAGCATTACTTC
AATCAAAGATGAAGACGGTAAGAAAGTCATCACAGGTAATGCCAGCTTGA
GTAAAGATGGTCTGCGCATATACGAGGTCTTCGATATAGCTATCAGCATC
GAAGAATCTGTATAA
pfaD
ATGAATCCTACAGCAACTAACGAAATGCTTTCTCCGTGGCCATGGGCTGT
GACAGAGTCAAATATCAGTTTTGACGTGCAAGTGATGGAACAACAACTTA
AAGATTTTAGCCGGGCATGTTACGTGGTCAATCATGCCGACCACGGCTTT
GGTATTGCGCAAACTGCCGATATCGTGACTGAACAAGCGGCAAACAGCAC
AGATTTACCTGTTAGTGCTTTTACTCCTGCATTAGGTACCGAAAGCCTAG
GCGACAATAATTTCCGCCGCGTTCACGGCGTTAAATACGCTTATTACGCA
GGCGCTATGGCAAACGGTATTTCATCTGAAGAGCTAGTGATTGCCCTAGG
TCAAGCTGGCATTTTGTGTGGTTCGTTTGGAGCAGCCGGTCTTATTCCAA
GTCGCGTTGAAGCGGCAATTAACCGTATTCAAGCAGCGCTGCCAAATGGC
CCTTATATGTTTAACCTTATCCATAGTCCTAGCGAGCCAGCATTAGAGCG
TGGCAGCGTAGAGCTATTTTTAAAGCATAAGGTACGCACCGTTGAAGCAT
CAGCTTTCTTAGGTCTAACACCACAAATCGTCTATTACCGTGCAGCAGGA
TTGAGCCGAGACGCACAAGGTAAAGTTGTGGTTGGTAACAAGGTTATCGC
TAAAGTAAGTCGCACCGAAGTGGCTGAAAAGTTTATGATGCCAGCGCCCG
CAAAAATGCTACAAAAACTAGTTGATGACGGTTCAATTACCGCTGAGCAA
ATGGAGCTGGCGCAACTTGTACCTATGGCTGACGACATCACTGCAGAGGC
CGATTCAGGTGGCCATACTGATAACCGTCCATTAGTAACATTGCTGCCAA
CCATTTTAGCGCTGAAAGAAGAAATTCAAGCTAAATACCAATACGACACT
CCTATTCGTGTCGGTTGTGGTGGCGGTGTGGGTACGCCTGATGCAGCGCT
GGCAACGTTTAACATGGGCGCGGCGTATATTGTTACCGGCTCTATCAACC
AAGCTTGTGTTGAAGCGGGCGCAAGTGATCACACTCGTAAATTACTTGCC
ACCACTGAAATGGCCGATGTGACTATGGCACCAGCTGCAGATATGTTCGA
GATGGGCGTAAAACTGCAGGTGGTTAAGCGCGGCACGCTATTCCCAATGC
GCGCTAACAAGCTATATGAGATCTACACCCGTTACGATTCAATCGAAGCG
ATCCCATTAGACGAGCGTGAAAAGCTTGAGAAACAAGTATTCCGCTCAAG
CCTAGATGAAATATGGGCAGGTACAGTGGCGCACTTTAACGAGCGCGACC
CTAAGCAAATCGAACGCGCAGAGGGTAACCCTAAGCGTAAAATGGCATTG
ATTTTCCGTTGGTACTTAGGTCTTTCTAGTCGCTGGTCAAACTCAGGCGA
AGTGGGTCGTGAAATGGATTATCAAATTTGGGCTGGCCCTGCTCTCGGTG
CATTTAACCAATGGGCAAAAGGCAGTTACTTAGATAACTATCAAGACCGA
AATGCCGTCGATTTGGCAAAGCACTTAATGTACGGCGCGGCTTACTTAAA
TCGTATTAACTCGCTAACGGCTCAAGGCGTTAAAGTGCCAGCACAGTTAC
TTCGCTGGAAGCCAAACCAAAGAATGGCCTAA
pfaE
ATGGTAAGAGGCTATTTGCGCGCTTTATTGTCACAACATAGTGAAATACG
CCCCAATGAATGGCGCTTTGAATATGGCGACAAAGGTAAGCCTAGATTGA
GTGATGCGCAATTTGCTCAAACCGGGGTCCACTTTAATGTGAGTCATAGT
GGAGATTGGCTATTAGTAGGCATTTGCACTGCTGATAATAAAGGCGCCAG
TCAGGCAAGCAAGGAGGAAACTGACTCTGCTAGTATTGAGTTTGGCGTCG
ACATTGAGCGTTGCCGTAACAGCACCAATATCCACTCTATTCTTAGTCAT
TATTTCTCTGAATCAGAAAAGCGAGCCTTGTTAGCGTTACCAGAGGCCTT
GCAGCGAGACCGCTTTTTTGATTTGTGGGCGCTCAAGGAGTCTTACATTA
AAGCGAAAGGACTTGGGCTGGCATTATCGCTAAAATCTTTTGCGTTTGAC
TTCTCTGCACTGAGCGAAACTTTTCTTGGAGTTAATGCACCTAAAAGCTT
GAGCCATTGTGTTGATATTTCCGATGCTATTGCGGATCACAAGGTTGAGC
ATCAACTTAATCAGCGACAGGTTTTGTTAAAACAAGATATTGGTCTTGCT
TTACTAGAGTCGAGTTCTAATAAGCCTAACGCTGAGCCACAAAAGTCTGG
TTTAGGTTTGATTGAGGCTAAAGAACAGCAAATGAACGCTGCTGATAATT
GGCATTGTTTACTGGGCCATCTTGATGATAGTTATCGTTTTGCACTGAGT
ATTGGTCAGTGTCAGCAAATAAGTATTGCAGCAGAAGAAGTGAATTTTAA
AGCTGTTGTTCGAGCTTCAGCTAAGACTAGCTAG

What is claimed is:

1. A proteinic biomass preparation comprising a non-native organism of the Clostridia class, which organism expresses
(i) a modified aspartate kinase characterized by reduced lysine inhibition, reduced threonine inhibition, and/or reduced methionine inhibition compared with the unmodified enzyme in native organism of the same genus and species;
(ii) a modified homoserine dehydrogenase characterized by reduced threonine inhibition compared with the unmodified enzyme in native organism of the same genus and species;
(iii) a modified homoserine kinase characterized by reduced methionine inhibition compared with the unmodified enzyme in native organism of the same genus and species;
(iv) a modified anthranilate synthase characterized by reduced tryptophan inhibition compared with the unmodified enzyme in native organism of the same genus and species;
(v) a functional lycopene pathway and the genes crtY, crtW, and crtZ and/or
(vi) a functional oleic acid pathway and the four gene operon (pfaABCD).

2-15. (canceled)

16. A preparation according to claim 1, wherein said non-native organism is an acetogen.

17-18. (canceled)

19. A preparation according to claim 1, comprising, on a dry basis, at least 55% wt protein.

20. A preparation according to claim 1, comprising at least one selected from the group consisting of:
on total protein content, at least 6% wt lysine;
(ii) on total protein content, at least 3% wt threonine;
(iii) on total protein content, at least 1.5% wt methionine;
(iv) on total protein content, at least 0.5% wt tryptophan;
(v) on a dry basis, at least 0.01% wt astaxanthin;
(vi) on a dry basis, at least 0.1% wt eicosapentaenoic acid; and
(vii) on a dry basis, at least 0.1% wt docosahexaenoic acid.

21-43. (canceled)

44. A proteinic biomass preparation comprising an organism of the Clostridia class, wherein said preparation comprises,
(i) on dry basis at least 55% wt protein;
(ii) on total protein content, at least 6% wt lysine;
(iii) on total protein content, at least 3% wt threonine;
(iv) on total protein content, at least 1.5% wt methionine;
(v) on total on total protein content, at least 0.5% wt tryptophan;
(vi) on a dry basis, at least 0.01% wt astaxanthin;
(vii) on a dry basis, at least 0.1wt eicosapentaenoic acid, and/or
(viii) on a dry basis, at least 0.1% wt docosahexaenoic acid.

45. (canceled)

46. A preparation according to claim 44, comprising (i) and at least one of (ii) to (v).

47-50. (canceled)

51. A preparation according to claim 44, wherein said organism expresses
(i) a modified aspartate kinase characterized by reduced lysine inhibition, reduced threonine inhibition, and/or reduced methionine inhibition compared with the unmodified enzyme in native organism of the same genus and species;
(ii) a modified homoserine dehydrogenase characterized by reduced threonine inhibition compared with the unmodified enzyme in native organism of the same genus and species;
(iii) a modified homoserine kinase characterized by reduced methionine inhibition compared with the unmodified enzyme in native organism of the same genus and species;
(iv) a modified anthranilate synthase characterized by reduced tryptophan inhibition compared with the unmodified enzyme in native organism of the same genus and species;
(v) a functional lycopene pathway and the genes crtY, crtW, and crtZ and/or
(vi) a functional oleic acid pathway and the four gene operon (pfaABCD)

52-55. (canceled)

56. A biomass according to claim 44, wherein said organism further expresses the gene pfaE.

57. A preparation according to claim 44, wherein said organism is selected from Butyribacterium methylotrophicum, Eubacterium limosum, and Clostridium kluyveri.

58. A preparation according to claim 44, wherein said organism is an acetogen.

59-61. (canceled)

62. A preparation according to claim 44, further comprising digestibility-enhancing enzymes selected from the group consisting of phytases, cellulases, lipases, amylases, arabinases, pectinases, mannases, keratinases, proteases, tannases, galactosidases, glucosidases, invertases and combinations thereof.

63. (canceled)

64. A preparation according to claim 51, wherein said non-native organism further expresses a diphosphate-fructose-6-phosphate 1-phosphotransferase (PFP, EC 2.7.1.90).

65. A preparation according to claim 44, wherein phosphofructokinase 1 (EC 2.7.1.11, pfkA, BUME_09340) has been deleted from the genome of said non-native organism.

66. A preparation according to claim 44, wherein acetyl-CoA acetyltransferase gene (th1A, EC 2.3.1.9, BUME_07140) been deleted from the genome of said non-native organism.

67. Animal feed comprising a preparation according to claim 44.

68. (canceled)

69. A method for producing of a biomass comprising culturing an organism according to claim 44 in a fermentation medium comprising a carbon source and a nitrogen source, whereby biomass is generated in a fermentation broth.

70. A method according to claim 69, wherein said culturing is anaerobic.

71-73. (canceled)

74. A method according to claim 69, wherein said non-native organism fixes CO₂.

75. (canceled)

76. A method according to claim 69, wherein biomass generation yield is greater than 35 gram biomass per 100 gram of carbon source consumed.


SEQUENCE LISTING (20 sequences)

SEQ ID NO: 1 - protein, 163 aa, Butyribacterium methylotrophicum
MTTTYMESHSVDGLTVDHKNLMISLKKVPVNSIILTRCLSELSDADVNVDIITQTAPVKNAFDVSFIVLERDLEKVKDIVNALGEEYPEIKITINKDITRLSVSGIGMRTQSGVAAKFFQVLADNDVQILMITTSEIRISCIIKIEDTEKAVAATKEAFDLED

SEQ ID NO: 2 - protein, 404 aa, Butyribacterium methylotrophicum
MSIIVQKYGGTSMGTIDRIKNVARRIIKKREDGNQMVVVVSAMGKSTDELVKMAYSISDAPPRRELDMLLATGEQVSISMLSMALNAMGYDAISFTGPQVGVHTMGHHGKSRIMDIETRKIEDALNDGKIVIIAGFQGVNENDDITTLGRGGSDTSAVALSCVLECPCEIYTDVDGIYGVDPRLYPPAKKLDTVSFDEMLEMASLGAGVMHARAIELGSKYNAEIYVASSIHDVPGTLIKEGDSNMSPMEQQAITGLAIDNDELMVSLKNVPFDMNITAQFFSDLAKKSINIDMISQTAPVYGAINISFSAPIEDLSELRKILYDFMEKYPQVEMDINKEISKLSVVGIGMRSQSGVAAKFFQLLADNNIPMLMITTSEIRISCVIPSELRDTAVMATADAFDL

SEQ ID NO: 3 - protein, 402 aa, Butyribacterium methylotrophicum
MEDLIVQKYGGTSVGTVEKIKRVARRIVETKNAGNKVVVVVSAMGKTTDELVDLALAINPNPPSREMDVLLATGEQVSISLLAMAIQTIGHDVVSLTGAQCGIQTSDVHKRARISGIDTARIERELADEKIVIVAGFQGVDENRDITTLGRGGSDTSAVAIAAALEAKCCEIYTDVDGVYNADPRVVPTATKMDEVSYQEVLEMASLGAGVLHPRSVELAEKFKMPLIVRSSYNNNEGTIIKEDVKMEKVLVRGIALDENIAKISIFEVPDQPGIAFKLFSMLASANIHVDMIVQNVNRTAVNDISFTVDADELQEAVEVSQKFAFEVEAQKVAFDKGVAKLSVVGTGIVANAEIASKFFESLFELGINIQTISTSEIKISCLIDKERAKEAMIHIHKKFDM

SEQ ID NO: 4 - protein, 414 aa, Butyribacterium methylotrophicum
MNIGLLGFGTIGTGVYELINLNKGRFAKNLDEKVVITKILDKDPNKKVAEEDKVARVVTNPDDIMDDPEIEIVIALLGGMDFEYGLIKRALQSGKHVVTANKAVISEYFEELLTIAAENNVILRYEASVGGGIPIIGSLKEELKINRVNEIKGILNGTTNFILSKMTEEGADFADTLKLAQSIGFAEADPTADIEGYDVSRKLAILSSLAYGGIIKDEDVRKRGLSDVRAVDIEMAGDYGYIIKYLGHSVLKEGNQVYTTVEPVMFKEASIMSNVNSEFNIISIVGDIIGELQFYGKGAGKDATANAVVGDALYIINCIKDNNFPKPLVFRKQLDKKGVGAFKGKYYLRVDIDSHETFEHALNAVDEVCARKNIIVSDNRVFFMTEPVEADVFDAMVAKIKEKQSECFYARIYE

SEQ ID NO: 5 - protein, 422 aa, Butyribacterium methylotrophicum
MVDGKFIEMQTEIMKNYPPANAQALLNIFNAASGMQEYLDDVLAKTRESYLYTQLRDLVENYYDIGTLLDVYQIFGGYINTSFGIYTEKNGEKQTWFVRKYKNGKELESLLFEHSMLKYARENGFSYGAVPIPAKDGKTYHEVTETTTEGETKSYFAVFNFVGGKAHYDWIPNWANAGVADLTVTSAAKSLAEFHNSTRGFDPEGRHGDNIMDNEDITVNEIIRKFPKTLKEYRKSYAEAGFENVYTEYYDANYDYFAKMCERSVIPDADYNTMVSNVCHCDFHPGNFKYLDNGEVCGSFDYDMAKIDSRLFELGLAIHYCFSSWLSDTNGIINLGRASLFVKTYDEELHKAGGIEPLTAIEKKYLYEVTIQGALYDLGWCSSACVYDSTLDPYEYLFYTQHFVACLKWLEANEEAFRKAFK

SEQ ID NO: 6 - protein, 297 aa, Butyribacterium methylotrophicum
MIKVRVPATTANIGPGFDAFGMAFQLYNIFSFEERDNGKLTIRGVERRYQGKSNLVYKAMLKVFNRVHYRPKGIYIYTDVNIPVSRGLGSSAACIVGGLVGANTLCGAPLTGKELFDMAVEMEGHPDNVAPAMFGGLVVSLGLKEENHYIKKEVSQCFEFYGLIPDFTLSTMEARKALPKKVFHKDAVFNVSRATMTYLALTEGRPDILKVSVEDKLHQPYREGLIAHYDEVSQKARELGALNTCISGAGPTLLAITTRDNDQFYAEMGKYLKEKLPGWTLLKLEPDNTGVCTDQHS

SEQ ID NO: 7 - protein, 488 aa, Butyribacterium methylotrophicum
MKIPSLETVQRQSEGFAAFPVSCEIFADIKTPIQVLKILKSISSRCYLLESVEGVEKWGRYSFLGFDPVVEVKCKDGLMEIKNGTAVRLETDDPGQEIRRILSEYRSPKVAELPPFTGGFVGYFSYDYLKYSEPSLRFDGDDSAGFNDLDLMLFEKVIAFDQLRQKIVIMVTIKTDHLAVNYNRAVRELDYLVGLIHSDVPASGQLPRLLSDFKPHFDREAYCEIVKKTKGYIREGDIFQAVPSNRLTAEMEGSLFNTYRVLRTINPSPYMYYIACDDLEIAGASPETLVKLQDGELSTFPIAGTSPRGRTSEEDAELEERLLKDPKELAEHNMLVDLGRNDLGRVSRYGSVKVQEYLKVQKYSHVMHITSVVTGQLRENMDQLDAVTAVLPAGTLSGAPKIRACEIINELEGIRRGIYGGAIGYIDFTGNMDVCIAIRTAVKKDGRVYVQSGGGVVADSDPEKEYQESINKAMAVVEAVKQSVEVMD

SEQ ID NO: 8 - protein, 193 aa, Butyribacterium methylotrophicum
MILIIDNYDSFSYNLVQQVGRVNPDLKVIRNDALSVDEIEALHPSHIILSPGPGRPADAGVCEAVVRRFSGKLPVLGVCLGHQAICEAFGAEVTYASELIHGKRSSIHIANGSPLFKGLPPIMDAARYHSLAVSRSSLPDELLIIAEDNADEVMGVKHRDFDVYGLQFHPESILTPQGNIIIENFLALGGKIQ

SEQ ID NO: 9 - protein, 573 aa, Unknown
  Description of Unknown Glb1 sequence
MVSARIVVLLAVLLCAAAAVASSWEDDNHHHHGGHKSGRCVRRCEDRPWHQRPRCLEQCREEEREKRQERSRHEADDRSGEGSSEDEREREQEKEEKQKDRRPYVFDRRSFRRVVRSEQGSLRVLRPFDEVSRLLRGIRDYRVAVLEANPRSFVVPSHTDAHCIGYVAEGEGVVTTIENGERRSYTIKQGHVFVAPAGAVTYLANTDGRKKLVITKILHTISVPGEFQFFFGPGGRNPESFLSSFSKSIQRAAYKTSSDRLERLFGRHGQDKGIIVRATEEQTRELRRHASEGGHGPHWPLPPFGESRGPYSLLDQRPSIANQHGQLYEADARSFHDLAEHDVSVSFANITAGSMSAPLYNTRSFKIAYVPNGKGYAEIVCPHRQSQGGESERERGKGRRSEEEEESSEEQEEVGQGYHTIRARLSPGTAFVVPAGHPFVAVASRDSNLQIVCFEVHADRNEKVFLAGADNVLQKLDRVAKALSFASKAEEVDEVLGSRREKGFLPGPKESGGHEEREQEEEEREERHGGRGERERHGREEREKEEEEREGRHGRGRREEVAETLLRMVTARM

SEQ ID NO: 10 - protein, 206 aa, Unknown
  Description of Unknown Glb3 sequence
MAKIAAAAAAALCFAALVAVAVCQGEVERQRLRDLQCWQEVQESPLDACRQVLDRQLTGGGGGGGVGPFRWGTGLRMRCCQQLQDVSRECRCAAIRSMVRGYEEAMPPLEKGWWPWGRQQQPPPQGGGGGQGGYYYPCSRPGEGYGYGQGGQRQMYPPCRPGTTGGGPRIGRVRLTKAREYAAGLPMMCRLSEPQECSIFSGGDQY

SEQ ID NO: 11 - protein, 518 aa, Avena sp.
MATTRFPSLLFYSCIFLLCNGSMAQLFGQSFTPWQSSRQGGLRGCKFDRLQAFEPLRQVRSQAGITEYFDEQNEQFRCAGVSVIRRVIEPQGLLLPQYHNAPGLVYILQGRGFTGLTFPGCPATFQQQFQQFDQARFAQGQSKSQNLKDEHQRVHHIKQGDVVALPAGIVHWCYNDGDAPIVAVYVFDVNNNANQLEPRQKEFLLAGNNKREQQFGQNIFSGFSVQLLSEALGISQQAAQKIQSQNDQRGEIIRVSQGLQFLKPFVSQQGPVEHQAYQPIQSQQEQSTQYQVGQSPQYQEGQSTQYQSGQSWDQSFNGLEENFCSLEARQNIENPKRADTYNPRAGRITHLNSKNFPTLNLVQMSATRVNLYQNAILSPYWNINAHSVMHMIQGRARVQVVNNHGQTVFNDILRRGQLLIIPQHYVVLKKAEREGCQYISFKTTPNSMVSYIAGKTSILRALPVDVLANAYRISRQESQNLKNNRGEEFGAFTPKFAQTGSQSYQDEGESSSTEKASE

SEQ ID NO: 12 - protein, 459 aa, Sesamum indicum
MVAFKFLLALSLSLLVSAAIAQTREPRLTQGQQCRFQRISGAQPSLRIQSEGGTTELWDERQEQFQCAGIVAMRSTIRPNGLSLPNYHPSPRLVYIERGQGLISIMVPGCAETYQVHRSQRTMERTEASEQQDRGSVRDLHQKVHRLRQGDIVAIPSGAAHWCYNDGSEDLVAVSINDVNHLSNQLDQKFRAFYLAGGVPRSGEQEQQARQTFHNIFRAFDAELLSEAFNVPQETIRRMQSEEEERGLIVMARERMTFVRPDEEEGEQEHRGRQLDNGLEETFCTMKFRTNVESRREADIFSRQAGRVHVVDRNKLPILKYMDLSAEKGNLYSNALVSPDWSMTGHTIVYVTRGDAQVQVVDHNGQALMNDRVNQGEMFVVPQYYTSTARAGNNGFEWVAFKTTGSPMRSPLAGYTSVIRAMPLQVITNSYQISPNQAQALKMNRGSQSFLLSPGGRRS

SEQ ID NO: 13 - DNA, 1203 bp, Artificial Sequence
  Description of Artificial Sequence Synthetic polynucleotide
atgaacgggcgcagggcagatctggcgattgttggcggcggcctgtcgggcggcctgatcgcgctggcgctgaggaacgcgcggcccgagctcgacgtcaggctgatagaagcgggcgagaggctgggcggcaatcaccgctggagctggttcgagaacgatctcggcaaaagcggcaacgaactcatgcagccgtttcgcaagaccgaatggcagggctacgatgtttatttcccgaaatattcccgccgtctgaaatctcgctactattctctggcatcgcccgatttcgacgccggattgcggcgggaattggcgcaagacactgttcataccggcaggaaagtggtggaatgcacgcctgacagtgtcacgctggaagggggagacgccataccggcccgtgccgtggtggattgccgagggttcgagccgagcgagctcgtgcgtggcggctggcaggtcttcatgggccgccacctgcgcacccatgccccgcacggcattacccgaccggtcatcatggacgtgaccgtagaacagctcgacggctaccgcttcgtttacgtcctcccgctcggcgcaagcgacatcttcatcgaggacacctattacaattccaaccccgagctggatcgcggcgcgctgtcgagccggctcgaccgatattgccgccagaacgggtgggaaggcgacatcgtcggcagcgagaccggggtcctgccggtcattaccggcggcgatttcgctgcctatcgccgcgagcgcgagctcgaccggatgccgcaggcgggtgcccgcgcgctgctggcccacccgctgaccagctacaccctgccgcaggcggtcgaaaccgcgcgcctgatcgcgcgcaacgcggacctgcctggcgaccagctggccgcgctgctcgccgcccatgcccagcggcactggaatcgcacgagctattaccgcctattgggcaggatacttttcgaagccccgcagcctaaggaacgctatcgaatcttcgagcgtttctacacactcgacgaggacttgatcgagcggttctatgccgcgcgttcgcgcccgcaggaaaaggcccgcgttttgtggggcaatccgccggtcgcgatccaccgcgccatcgcggcgatcttcagcaagagtgcgccgctggtggtgccggacagaacgaaaggtcgcgtgtcgacatga

SEQ ID NO: 14 - DNA, 768 bp, Artificial Sequence
  Description of Artificial Sequence Synthetic polynucleotide
atgtctgacggtcctgccttttcgttgcctgcgcgctcgcgtcagcaggcgatcgggttgacgctcgccgtgctgatcgctggcgcgtggctcggcatccacgcctatgcgatgttcgttttcgagctgagttggcagaacctgcccttcgcacttctgctagcaacggtgcaaacgtggctgtcggttggcgtcttcatcgttagtcatgacgcaatgcacggctcgctggcacccggcaggccgacgctgaattcggcgatcggcgcgtttctcttggccctttacgcaggcttcggctggcgacgaatgcgtgacgcgcattttacccaccacaagctggcgggccatgcgggagacccggatttcgacgagcacaatccgcgcagtttctggcgctggtacggaaccttcttccggcgctatttcagctggcaatcgatcctgttcgtgcatgtcgttgtcggcatctactggctagtcctcgacattccgatggtgcagatcgtcctgctctacggcgcaccggcactgctttcatcgctgcagcttttctacttcgggacctttcgtccacatcggcgttccgaagagggttttgccgaccggcacaacgcgcgcagcgatggcttcggcacgttggccagcctcgccacgtgcttccatttcggctatcatctcgaacatcatcggcgtcccgacgtgccgtggtgggccttgcccggcgcgcgagaagcggggatcggaatggaagcgcaaaacgcatga

SEQ ID NO: 15 - DNA, 501 bp, Artificial Sequence
  Description of Artificial Sequence Synthetic polynucleotide
atgagctggtgggcaatcgccctgatcgtattcggtgccgtggtcggcatggagttttttgcctggtttgcccacaaatacatcatgcacggctggggctggtcctggcatcgcgaccatcatgagccgcacgacaacacactggaaaagaacgatcttttcgcggtcgtgttcggatccgtagcggcgctgctttttgttataggcgccctgtggtccgatcctttgtggtgggctgcggtcgggatcacgctttacggcgtgatttatacgctggtgcacgacgggctggtccatcagcgctactggcgctggacgcccaagcgcggttacgcgaagcggctggtccaggcccaccggctgcatcatgccacggtaggcaaggagggcggggtgagtttcggcttcgttttcgcgcgcgatccggcaaaactgaaggctgaactcaagcagcagcgcgaacagggtctcgcggtcgtgcgcgacagcatgggcgcctga

SEQ ID NO: 16 - DNA, 8271 bp, Artificial Sequence
  Description of Artificial Sequence Synthetic polynucleotide
atgagccagacctctaaacctacaaactcagcaactgagcaagcacaagactcacaagctgactctcgtttaaataaacgactaaaagatatgccaattgctattgttggcatggcgagtatttttgcaaactctcgctatttgaataagttttgggacttaatcagcgaaaaaattgatgcgattactgaattaccatcaactcactggcagcctgaagaatattacgacgcagataaaaccgcagcagacaaaagctactgtaaacgtggtggctttttgccagatgtagacttcaacccaatggagtttggcctgccgccaaacattttggaactgaccgattcatcgcaactattatcactcatcgttgctaaagaagtgttggctgatgctaacttacctgagaattacgaccgcgataaaattggtatcaccttaggtgtcggcggtggtcaaaaaattagccacagcctaacagcgcgtctgcaatacccagtattgaagaaagtattcgccaatagcggcattagtgacaccgacagcgaaatgcttatcaagaaattccaagaccaatatgtacactgggaagaaaactcgttcccaggttcacttggtaacgttattgcgggccgtatcgccaaccgcttcgattttggcggcatgaactgtgtggttgatgctgcctgtgctggatcacttgctgctatgcgtatggcgctaacagagctaactgaaggtcgctctgaaatgatgatcaccggtggtgtgtgtactgataactcaccctctatgtatatgagcttttcaaaaacgcccgcctttaccactaacgaaaccattcagccatttgatatcgactcaaaaggcatgatgattggtgaaggtattggcatggtggcgctaaagcgtcttgaagatgcagagcgcgatggcgaccgcatttactctgtaattaaaggtgtgggtgcatcatctgacggtaagtttaaatcaatctatgcccctcgcccatcaggccaagctaaagcacttaaccgtgcctatgatgacgcaggttttgcgccgcataccttaggtctaattgaagctcacggaacaggtactgcagcaggtgacgcggcagagtttgccggcctttgctcagtatttgctgaaggcaacgataccaagcaacacattgcgctaggttcagttaaatcacaaattggtcatactaaatcaactgcaggtacagcaggtttaattaaagctgctcttgctttgcatcacaaggtactgccgccgaccattaacgttagtcagccaagccctaaacttgatatcgaaaactcaccgttttatctaaacactgagactcgtccatggttaccacgtgttgatggtacgccgcgccgcgcgggtattagctcatttggttttggtggcactaacttccattttgtactagaagagtacaaccaagaacacagccgtactgatagcgaaaaagctaagtatcgtcaacgccaagtggcgcaaagcttccttgttagcgcaagcgataaagcatcgctaattaacgagttaaacgtactagcagcatctgcaagccaagctgagtttatcctcaaagatgcagcagcaaactatggcgtacgtgagcttgataaaaatgcaccacggatcggtttagttgcaaacacagctgaagagttagcaggcctaattaagcaagcacttgccaaactagcagctagcgatgataacgcatggcagctacctggtggcactagctaccgcgccgctgcagtagaaggtaaagttgccgcactgtttgctggccaaggttcacaatatctcaatatgggccgtgaccttacttgttattacccagagatgcgtcagcaatttgtaactgcagataaagtatttgccgcaaatgataaaacgccgttatcgcaaactctgtatccaaagcctgtatttaataaagatgaattaaaggctcaagaagccattttgaccaataccgccaatgcccaaagcgcaattggtgcgatttcaatgggtcaatacgatttgtttactgcggctggctttaatgccgacatggttgcaggccatagctttggtgagctaagtgcactgtgtgctgcaggtgttatttcagctgatgactactacaagctggcttttgctcgtggtgaggctatggcaacaaaagcaccggctaaagacggcgttgaagcagatgcaggagcaatgtttgcaatcataaccaagagtgctgcagaccttgaaaccgttgaagccaccatcgctaaatttgatggggtgaaagtcgctaactataacgcgccaacgcaatcagtaattgcaggcccaacagcaactaccgctgatgcggctaaagcgctaactgagcttggttacaaagcgattaacctgccagtatcaggtgcattccacactgaacttgttggtcacgctcaagcgccatttgctaaagcgattgacgcagccaaatttactaaaacaagccgagcactttactcaaatgcaactggcggactttatgaaagcactgctgcaaagattaaagcctcgtttaagaaacatatgcttcaatcagtgcgctttactagccagctagaagccatgtacaacgacggcgcccgtgtatttgttgaatttggtccaaagaacatcttacaaaaattagttcaaggcacgcttgtcaacactgaaaatgaagtttgcactatctctatcaaccctaatcctaaagttgatagtgatctgcagcttaagcaagcagcaatgcagctagcggttactggtgtggtactcagtgaaattgacccataccaagccgatattgccgcaccagcgaaaaagtcgccaatgagcatttcgcttaatgctgctaaccatatcagcaaagcaactcgcgctaagatggccaagtctttagagacaggtatcgtcacctcgcaaatagaacatgttattgaagaaaaaatcgttgaagttgagaaactggttgaagtcgaaaagatcgtcgaaaaagtggttgaagtagagaaagttgttgaggttgaagctcctgttaattcagtgcaagccaatgcaattcaaacccgttcagttgtcgctccagtaatagagaaccaagtcgtgtctaaaaacagtaagccagcagtccagagcattagtggtgatgcactcagcaacttttttgctgcacagcagcaaaccgcacagttgcatcagcagttcttagctattccgcagcaatatggtgagacgttcactacgctgatgaccgagcaagctaaactggcaagttctggtgttgcaattccagagagtctgcaacgctcaatggagcaattccaccaactacaagcgcaaacactacaaagccacacccagttccttgagatgcaagcgggtagcaacattgcagcgttaaacctactcaatagcagccaagcaacttacgctccagccattcacaatgaagcgattcaaagccaagtggttcaaagccaaactgcagtccagccagtaatttcaacacaagttaaccatgtgtcagagcagccaactcaagctccagctccaaaagcgcagccagcacctgtgacaactgcagttcaaactgctccggcacaagttgttcgtcaagccgcaccagttcaagccgctattgaaccgattaatacaagtgttgcgactacaacgccttcagccttcagcgccgaaacagccctgagcgcaacaaaagtccaagccactatgcttgaagtggttgctgagaaaaccggttacccaactgaaatgctagagcttgaaatggatatggaagccgatttaggcatcgattctatcaagcgtgtagaaattcttggcacagtacaagatgagctaccgggtctacctgagcttagccctgaagatctagctgagtgtcgaacgctaggcgaaatcgttgactatatgggcagtaaactgccggctgaaggctctatgaattctcagctgtctacaggttccgcagctgcgactcctgcagcgaatggtctttctgcggagaaagttcaagcgactatgatgtctgtggttgccgaaaagactggctacccaactgaaatgctagagcttgaaatggatatggaagccgatttaggcatagattctatcaagcgcgttgaaattcttggcacagtacaagatgagctaccgggtctacctgagcttagccctgaagatctagctgagtgtcgtactctaggcgaaatcgttgactatatgaactctaaactcgctgacggctctaagctgccggctgaaggctctatgaattctcagctgtctacaagtgccgcagctgcgactcctgcagcgaatggtctctctgcggagaaagttcaagcgactatgatgtctgtggttgccgaaaagactggctacccaactgaaatgctagaacttgaaatggatatggaagctgaccttggcatcgattcaatcaagcgcgttgaaattcttggcacagtacaagatgagctaccgggtttacctgagctaaatccagaagatttggcagagtgtcgtactcttggcgaaatcgtgacttatatgaactctaaactcgctgacggctctaagctgccagctgaaggctctatgcactatcagctgtctacaagtaccgctgctgcgactcctgtagcgaatggtctctctgcagaaaaagttcaagcgaccatgatgtctgtagttgcagataaaactggctacccaactgaaatgcttgaacttgaaatggatatggaagccgatttaggtatcgattctatcaagcgcgttgaaattcttggcacagtacaagatgagctaccgggtttacctgagctaaatccagaagatctagcagagtgtcgcaccctaggcgaaatcgttgactatatgggcagtaaactgccggctgaaggctctgctaatacaagtgccgctgcgtctcttaatgttagtgccgttgcggcgcctcaagctgctgcgactcctgtatcgaacggtctctctgcagagaaagtgcaaagcactatgatgtcagtagttgcagaaaagaccggctacccaactgaaatgctagaacttggcatggatatggaagccgatttaggtatcgactcaattaaacgcgttgagattcttggcacagtacaagatgagctaccgggtctaccagagcttaatcctgaagatttagctgagtgccgtacgctgggcgaaatcgttgactatatgaactctaagctggctgacggctctaagcttccagctgaaggctctgctaatacaagtgccactgctgcgactcctgcagtgaatggtctttctgctgacaaggtacaggcgactatgatgtctgtagttgctgaaaagaccggctacccaactgaaatgctagaacttggcatggatatggaagcagaccttggtattgattctattaagcgcgttgaaattcttggcacagtacaagatgagctcccaggtttacctgagcttaatcctgaagatctcgctgagtgccgcacgcttggcgaaatcgttagctatatgaactctcaactggctgatggctctaaactttctacaagtgcggctgaaggctctgctgatacaagtgctgcaaatgctgcaaagccggcagcaatttcggcagaaccaagtgttgagcttcctcctcatagcgaggtagcgctaaaaaagcttaatgcggcgaacaagctagaaaattgtttcgccgcagacgcaagtgttgtgattaacgatgatggtcacaacgcaggcgttttagctgagaaacttattaaacaaggcctaaaagtagccgttgtgcgtttaccgaaaggtcagcctcaatcgccactttcaagcgatgttgctagctttgagcttgcctcaagccaagaatctgagcttgaagccagtatcactgcagttatcgcgcagattgaaactcaggttggcgctattggtggctttattcacttgcaaccagaagcgaatacagaagagcaaacggcagtaaacctagatgcgcaaagttttactcacgttagcaatgcgttcttgtgggccaaattattgcaaccaaagctcgttgctggagcagatgcgcgtcgctgttttgtaacagtaagccgtatcgacggtggctttggttacctaaatactgacgccctaaaagatgctgagctaaaccaagcagcattagctggtttaactaaaaccttaagccatgaatggccacaagtgttctgtcgcgcgctagatattgcaacagatgttgatgcaacccatcttgctgatgcaatcaccagtgaactatttgatagccaagctcagctacctgaagtgggcttaagcttaattgatggcaaagttaaccgcgtaactctagttgctgctgaagctgcagataaaacagcaaaagcagagcttaacagcacagataaaatcttagtgactggtggggcaaaaggggtgacatttgaatgtgcactggcattagcatctcgcagccagtctcactttatcttagctgggcgcagtgaattacaagctttaccaagctgggctgagggtaagcaaactagcgagctaaaatcagctgcaatcgcacatattatttctactggtcaaaagccaacgcctaagcaagttgaagccgctgtgtggccagtgcaaagcagcattgaaattaatgccgccctagccgcctttaacaaagttggcgcctcagctgaatacgtcagcatggatgttaccgatagcgccgcaatcacagcagcacttaatggtcgctcaaatgagatcaccggtcttattcatggcgcaggtgtactagccgacaagcatattcaagacaagactcttgctgaacttgctaaagtttatggcactaaagtcaacggcctaaaagcgctgctcgcggcacttgagccaagcaaaattaaattacttgctatgttctcatctgcagcaggtttttacggtaatatcggccaaagcgattacgcgatgtcgaacgatattcttaacaaggcagcgctgcagttcaccgctcgcaacccacaagctaaagtcatgagctttaactggggtccttgggatggcggcatggttaacccagcgcttaaaaagatgtttaccgagcgtggtgtgtacgttattccactaaaagcaggtgcagagctatttgccactcagctattggctgaaactggcgtgcagttgctcattggtacgtcaatgcaaggtggcagcgacactaaagcaactgagactgcttctgtaaaaaagcttaatgcgggtgaggtgctaagtgcatcgcatccgcgtgctggtgcacaaaaaacaccactacaagctgtcactgcaacgcgtctgttaaccccaagtgccatggtcttcattgaagatcaccgcattggcggtaacagtgtgttgccaacggtatgcgccatcgactggatgcgtgaagcggcaagcgacatgcttggcgctcaagttaaggtacttgattacaagctattaaaaggcattgtatttgagactgatgagccgcaagagttaacacttgagctaacgccagacgattcagacgaagctacgctacaagcattaatcagctgtaatgggcgtccgcaatacaaggcgacgcttatcagtgataatgccgatattaagcaacttaacaagcagtttgatttaagcgctaaggcgattaccacagcaaaagagctttatagcaacggcaccttgttccacggtccgcgtctacaagggatccaatctgtagtgcagttcgatgatcaaggcttaattgctaaagtcgctctgcctaaggttgaacttagcgattgtggtgagttcttgccgcaaacccacatgggtggcagtcaaccttttgctgaggacttgctattacaagctatgctggtttgggctcgccttaaaactggctcggcaagtttgccatcaagcattggtgagtttacctcataccaaccaatggcctttggtgaaactggtaccatagagcttgaagtgattaagcacaacaaacgctcacttgaagcgaatgttgcgctatatcgtgacaacggcgagttaagtgccatgtttaagtcagctaaaatcaccattagcaaaagcttaaattcagcatttttacctgctgtcttagcaaacgacagtgaggcgaattag

SEQ ID NO: 17 - DNA, 2343 bp, Artificial Sequence
  Description of Artificial Sequence Synthetic polynucleotide
gtggaacaaacgcctaaagctagtgcgatgccgctgcgcatcgcacttatcttactgccaacaccgcagtttgaagttaactctgtcgaccagtcagtattagccagctatcaaacactgcagcctgagctaaatgccctgcttaatagtgcgccgacacctgaaatgctcagcatcactatctcagatgatagcgatgcaaacagctttgagtcgcagctaaatgctgcgaccaacgcaattaacaatggctatatcgtcaagcttgctacggcaactcacgctttgttaatgctgcctgcattaaaagcggcgcaaatgcggatccatcctcatgcgcagcttgccgctatgcagcaagctaaatcgacgccaatgagtcaagtatctggtgagctaaagcttggcgctaatgcgctaagcctagctcagactaatgcgctgtctcatgctttaagccaagccaagcgtaacttaactgatgtcagcgtgaatgagtgttttgagaacctcaaaagtgaacagcagttcacagaggtttattcgcttattcagcaacttgctagccgcacccatgtgagaaaagaggttaatcaaggtgtggaacttggccctaaacaagccaaaagccactattggtttagcgaatttcaccaaaaccgtgttgctgccatcaactttattaatggccaacaagcaaccagctatgtgcttactcaaggttcaggattgttagctgcgaaatcaatgctaaaccagcaaagattaatgtttatcttgccgggtaacagtcagcaacaaataaccgcatcaataactcagttaatgcagcaattagagcgtttgcaggtaactgaggttaatgagctttctctagaatgccaactagagctgctcagcataatgtatgacaacttagtcaacgcagacaaactcactactcgcgatagtaagcccgcttatcaggctgtgattcaagcaagctctgttagcgctgcaaagcaagagttaagcgcgcttaacgatgcactcacagcgctgtttgctgagcaaacaaacgccacatcaacgaataaaggcttaatccaatacaaaacaccggcgggcagttacttaaccctaacaccgcttggcagcaacaatgacaacgcccaagcgggtcttgcttttgtctatccgggtgtgggaacggtttacgccgatatgcttaatgagctgcatcagtacttccctgcgctttacgccaaacttgagcgtgaaggcgatttaaaggcgatgctacaagcagaagatatctatcatcttgaccctaaacatgctgcccaaatgagcttaggtgacttagccattgctggcgtggggagcagctacctgttaactcagctgctcaccgatgagtttaatattaagcctaattttgcattaggttactcaatgggtgaagcatcaatgtgggcaagcttaggcgtatggcaaaacccgcatgcgctgatcagcaaaacccaaaccgacccgctatttacttctgctatttccggcaaattgaccgcggttagacaagcttggcagcttgatgataccgcagcggaaatccagtggaatagctttgtggttagaagtgaagcagcgccgattgaagccttgctaaaagattacccacacgcttacctcgcgattattcaaggggatacctgcgtaatcgctggctgtgaaatccaatgtaaagcgctacttgcagcactgggtaaacgcggtattgcagctaatcgtgtaacggcgatgcatacgcagcctgcgatgcaagagcatcaaaatgtgatggatttttatctgcaaccgttaaaagcagagcttcctagtgaaataagctttatcagcgccgctgatttaactgccaagcaaacggtgagtgagcaagcacttagcagccaagtcgttgctcagtctattgccgacaccttctgccaaaccttggactttaccgcgctagtacatcacgcccaacatcaaggcgctaagctgtttgttgaaattggcgcggatagacaaaactgcaccttgatagacaagattgttaaacaagatggtgccagcagtgtacaacatcaaccttgttgcacagtgcctatgaacgcaaaaggtagccaagatattaccagcgtgattaaagcgcttggccaattaattagccatcaggtgccattatcggtgcaaccatttattgatggactcaagcgcgagctaacactttgccaattgaccagccaacagctggcagcacatgcaaatgttgacagcaagtttgagtctaaccaagaccatttacttcaaggggaagtctaa

SEQ ID NO: 18 - DNA, 6015 bp, Artificial Sequence
  Description of Artificial Sequence Synthetic polynucleotide
atgtcattaccagacaatgcttctaaccacctttctgccaaccagaaaggcgcatctcaggcaagtaaaaccagtaagcaaagcaaaatcgccattgtcggtttagccactctgtatccagacgctaaaaccccgcaagaattttggcagaatttgctggataaacgcgactctcgcagcaccttaactaacgaaaaactcggcgctaacagccaagattatcaaggtgtgcaaggccaatctgaccgtttttattgtaataaaggcggctacattgagaacttcagctttaatgctgcaggctacaaattgccggagcaaagcttaaatggcttggacgacagcttcctttgggcgctcgatactagccgtaacgcactaattgatgctggtattgatatcaacggcgctgatttaagccgcgcaggtgtagtcatgggcgcgctgtcgttcccaactacccgctcaaacgatctgtttttgccaatttatcacagcgccgttgaaaaagccctgcaagataaactaggcgtaaaggcatttaagctaagcccaactaatgctcataccgctcgcgcggcaaatgagagcagcctaaatgcagccaatggtgccattgcccataacagctcaaaagtggtggccgatgcacttggccttggcggcgcacaactaagcctagatgctgcctgtgctagttcggtttactcattaaagcttgcctgcgattacctaagcactggcaaagccgatatcatgctagcaggcgcagtatctggcgcggatcctttctttattaatatgggattctcaatcttccacgcctacccagaccatggtatctcagtaccgtttgatgccagcagtaaaggtttgtttgctggcgaaggcgctggcgtattagtgcttaaacgtcttgaagatgccgagcgcgacaatgacaaaatctatgcggttgttagcggcgtaggtctatcaaacgacggtaaaggccagtttgtattaagccctaatccaaaaggtcaggtgaaggcctttgaacgtgcttatgctgccagtgacattgagccaaaagacattgaagtgattgagtgccacgcaacaggcacaccgcttggcgataaaattgagctcacttcaatggaaaccttctttgaagacaagctgcaaggcaccgatgcaccgttaattggctcagctaagtctaacttaggccacctattaactgcagcgcatgcggggatcatgaagatgatcttcgccatgaaagaaggttacctgccgccaagtatcaatattagtgatgctatcgcttcgccgaaaaaactcttcggtaaaccaaccctgcctagcatggttcaaggctggccagataagccatcgaataatcattttggtgtaagaacccgtcacgcaggcgtatcggtatttggctttggtggctgtaacgcccatctgttgcttgagtcatacaacggcaaaggaacagtaaaggcagaagccactcaagtaccgcgtcaagctgagccgctaaaagtggttggccttgcctcgcactttgggcctcttagcagcattaatgcactcaacaatgctgtgacccaagatgggaatggctttatcgaactgccgaaaaagcgctggaaaggccttgaaaagcacagtgaactgttagctgaatttggcttagcatctgcgccaaaaggtgcttatgttgataacttcgagctggactttttacgctttaaactgccgccaaacgaagatgaccgtttgatctcacagcagctaatgctaatgcgagtaacagacgaagccattcgtgatgccaagcttgagccggggcaaaaagtagctgtattagtggcaatggaaactgagcttgaactgcatcagttccgcggccgggttaacttgcatactcaattagcgcaaagtcttgccgccatgggcgtgagtttatcaacggatgaataccaagcgcttgaagccatcgccatggacagcgtgcttgatgctgccaagctcaatcagtacaccagctttattggtaatattatggcgtcacgcgtggcgtcactatgggactttaatggcccagccttcactatttcagcagcagagcaatctgtgagccgctgtatcgatgtggcgcaaaacctcatcatggaggataacctagatgcggtggtgattgcagcggtcgatctctctggtagctttgagcaagtcattcttaaaaatgccattgcacctgtagccattgagccaaacctcgaagcaagccttaatccaacatcagcaagctggaatgtcggtgaaggtgctggcgcggtcgtgcttgttaaaaatgaagctacatcgggctgctcatacggccaaattgatgcacttggctttgctaaaactgccgaaacagcgttggctaccgacaagctactgagccaaactgccacagactttaataaggttaaagtgattgaaactatggcagcgcctgctagccaaattcaattagcgccaatagttagctctcaagtgactcacactgctgcagagcagcgtgttggtcactgctttgctgcagcgggtatggcaagcctattacacggcttacttaacttaaatactgtagcccaaaccaataaagccaattgcgcgcttatcaacaatatcagtgaaaaccaattatcacagctgttgattagccaaacagcgagcgaacaacaagcattaaccgcgcgtttaagcaatgagcttaaatccgatgctaaacaccaactggttaagcaagtcaccttaggtggccgtgatatctaccagcatattgttgatacaccgcttgcaagccttgaaagcattactcagaaattggcgcaagcgacagcatcgacagtggtcaaccaagttaaacctattaaggccgctggctcagtcgaaatggctaactcattcgaaacggaaagctcagcagagccacaaataacaattgcagcacaacagactgcaaacattggcgtcaccgctcaggcaaccaaacgtgaattaggtaccccaccaatgacaacaaataccattgctaatacagcaaataatttagacaagactcttgagactgttgctggcaatactgttgctagcaaggttggctctggcgacatagtcaattttcaacagaaccaacaattggctcaacaagctcacctcgcctttcttgaaagccgcagtgcgggtatgaaggtggctgatgctttattgaagcaacagctagctcaagtaacaggccaaactatcgataatcaggccctcgatactcaagccgtcgatactcaaacaagcgagaatgtagcgattgccgcagaatcaccagttcaagttacaacacctgttcaagttacaacacctgttcaaatcagtgttgtggagttaaaaccagatcacgctaatgtgccaccatacacgccgccagtgcctgcattaaagccgtgtatctggaactatgccgatttagttgagtacgcagaaggcgatatcgccaaggtatttggcagtgattatgccattatcgacagctactcgcgccgcgtacgtctaccgaccactgactacctgttggtatcgcgcgtgaccaaacttgatgcgaccatcaatcaatttaagccatgctcaatgaccactgagtacgacatccctgttgatgcgccgtacttagtagacggacaaatcccttgggcggtagcagtagaatcaggccaatgtgacttgatgcttattagctatctcggtatcgactttgagaacaaaggcgagcgggtttatcgactactcgattgtaccctcaccttcctaggcgacttgccacgtggcggagataccctacgttacgacattaagatcaataactatgctcgcaacggcgacaccctgctgttcttcttctcgtatgagtgttttgttggcgacaagatgatcctcaagatggatggcggctgcgctggcttcttcactgatgaagagcttgccgacggtaaaggcgtgattcgcacagaagaagagattaaagctcgcagcctagtgcaaaagcaacgctttaatccgttactagattgtcctaaaacccaatttagttatggtgatattcataagctattaactgctgatattgagggttgttttggcccaagccacagtggcgtccaccagccgtcactttgtttcgcatctgaaaaattcttgatgattgaacaagtcagcaaggttgatcgcactggcggtacttggggacttggcttaattgagggtcataagcagcttgaagcagaccactggtacttcccatgtcatttcaagggcgaccaagtgatggctggctcgctaatggctgaaggttgtggccagttattgcagttctatatgctgcaccttggtatgcatacccaaactaaaaatggtcgtttccaacctcttgaaaacgcctcacagcaagtacgctgtcgcggtcaagtgctgccacaatcaggcgtgctaacttaccgtatggaagtgactgaaatcggtttcagtccacgcccatatgctaaagctaacatcgatatcttgcttaatggcaaagcggtagtggatttccaaaacctaggggtgatgataaaagaggaagatgagtgtactcgttatccacttttgactgaatcaacaacggctagcactgcacaagtaaacgctcaaacaagtgcgaaaaaggtatacaagccagcatcagtcaatgcgccattaatggcacaaattcctgatctgactaaagagccaaacaagggcgttattccgatttcccatgttgaagcaccaattacgccagactacccgaaccgtgtacctgatacagtgccattcacgccgtatcacatgtttgagtttgctacaggcaatatcgaaaactgtttcgggccagagttctcaatctatcgcggcatgatcccaccacgtacaccatgcggtgacttacaagtgaccacacgtgtgattgaagttaacggtaagcgtggcgactttaaaaagccatcatcgtgtatcgctgaatatgaagtgcctgcagatgcgtggtatttcgataaaaacagccacggcgcagtgatgccatattcaattttaatggagatctcactgcaacctaacggctttatctcaggttacatgggcacaaccctaggcttccctggccttgagctgttcttccgtaacttagacggtagcggtgagttactacgtgaagtagatttacgtggtaaaaccatccgtaacgactcacgtttattatcaacagtgatggccggcactaacatcatccaaagctttagcttcgagctaagcactgacggtgagcctttctatcgcggcactgcggtatttggctattttaaaggtgacgcacttaaagatcagctaggcctagataacggtaaagtcactcagccatggcatgtagctaacggcgttgctgcaagcactaaggtgaacctgcttgataagagctgccgtcactttaatgcgccagctaaccagccacactatcgtctagccggtggtcagctgaactttatcgacagtgttgaaattgttgataatggcggcaccgaaggtttaggttacttgtatgccgagcgcaccattgacccaagtgattggttcttccagttccacttccaccaagatccggttatgccaggctccttaggtgttgaagcaattattgaaaccatgcaagcttacgctattagtaaagacttgggcgcagatttcaaaaatcctaagtttggtcagattttatcgaacatcaagtggaagtatcgcggtcaaatcaatccgctgaacaagcagatgtctatggatgtcagcattacttcaatcaaagatgaagacggtaagaaagtcatcacaggtaatgccagcttgagtaaagatggtctgcgcatatacgaggtcttcgatatagctatcagcatcgaagaatctgtataa

SEQ ID NO: 19 - DNA, 1632 bp, Artificial Sequence
  Description of Artificial Sequence Synthetic polynucleotide
atgaatcctacagcaactaacgaaatgctttctccgtggccatgggctgtgacagagtcaaatatcagttttgacgtgcaagtgatggaacaacaacttaaagattttagccgggcatgttacgtggtcaatcatgccgaccacggctttggtattgcgcaaactgccgatatcgtgactgaacaagcggcaaacagcacagatttacctgttagtgcttttactcctgcattaggtaccgaaagcctaggcgacaataatttccgccgcgttcacggcgttaaatacgcttattacgcaggcgctatggcaaacggtatttcatctgaagagctagtgattgccctaggtcaagctggcattttgtgtggttcgtttggagcagccggtcttattccaagtcgcgttgaagcggcaattaaccgtattcaagcagcgctgccaaatggcccttatatgtttaaccttatccatagtcctagcgagccagcattagagcgtggcagcgtagagctatttttaaagcataaggtacgcaccgttgaagcatcagctttcttaggtctaacaccacaaatcgtctattaccgtgcagcaggattgagccgagacgcacaaggtaaagttgtggttggtaacaaggttatcgctaaagtaagtcgcaccgaagtggctgaaaagtttatgatgccagcgcccgcaaaaatgctacaaaaactagttgatgacggttcaattaccgctgagcaaatggagctggcgcaacttgtacctatggctgacgacatcactgcagaggccgattcaggtggccatactgataaccgtccattagtaacattgctgccaaccattttagcgctgaaagaagaaattcaagctaaataccaatacgacactcctattcgtgtcggttgtggtggcggtgtgggtacgcctgatgcagcgctggcaacgtttaacatgggcgcggcgtatattgttaccggctctatcaaccaagcttgtgttgaagcgggcgcaagtgatcacactcgtaaattacttgccaccactgaaatggccgatgtgactatggcaccagctgcagatatgttcgagatgggcgtaaaactgcaggtggttaagcgcggcacgctattcccaatgcgcgctaacaagctatatgagatctacacccgttacgattcaatcgaagcgatcccattagacgagcgtgaaaagcttgagaaacaagtattccgctcaagcctagatgaaatatgggcaggtacagtggcgcactttaacgagcgcgaccctaagcaaatcgaacgcgcagagggtaaccctaagcgtaaaatggcattgattttccgttggtacttaggtctttctagtcgctggtcaaactcaggcgaagtgggtcgtgaaatggattatcaaatttgggctggccctgctctcggtgcatttaaccaatgggcaaaaggcagttacttagataactatcaagaccgaaatgccgtcgatttggcaaagcacttaatgtacggcgcggcttacttaaatcgtattaactcgctaacggctcaaggcgttaaagtgccagcacagttacttcgctggaagccaaaccaaagaatggcctaa

SEQ ID NO: 20 - DNA, 834 bp, Artificial Sequence
  Description of Artificial Sequence Synthetic polynucleotide
atggtaagaggctatttgcgcgctttattgtcacaacatagtgaaatacgccccaatgaatggcgctttgaatatggcgacaaaggtaagcctagattgagtgatgcgcaatttgctcaaaccggggtccactttaatgtgagtcatagtggagattggctattagtaggcatttgcactgctgataataaaggcgccagtcaggcaagcaaggaggaaactgactctgctagtattgagtttggcgtcgacattgagcgttgccgtaacagcaccaatatccactctattcttagtcattatttctctgaatcagaaaagcgagccttgttagcgttaccagaggccttgcagcgagaccgcttttttgatttgtgggcgctcaaggagtcttacattaaagcgaaaggacttgggctggcattatcgctaaaatcttttgcgtttgacttctctgcactgagcgaaacttttcttggagttaatgcacctaaaagcttgagccattgtgttgatatttccgatgctattgcggatcacaaggttgagcatcaacttaatcagcgacaggttttgttaaaacaagatattggtcttgctttactagagtcgagttctaataagcctaacgctgagccacaaaagtctggtttaggtttgattgaggctaaagaacagcaaatgaacgctgctgataattggcattgtttactgggccatcttgatgatagttatcgttttgcactgagtattggtcagtgtcagcaaataagtattgcagcagaagaagtgaattttaaagctgttgttcgagcttcagctaagactagctag